(19) United States
(12) Patent Application Publication
(10) Pub. No.: US 2011/0097418 A1
(43) Pub. Date: Apr. 28, 2011

(54) COMPOSITIONS AND METHODS FOR TREATING INFLUENZA

(75) Inventors: Francisco Diaz-Mitoma, Ottawa (CA); Andrei Ogrel, Russell (CA); Jose V. Torres, Davis, CA (US); David E. Anderson, Boston, MA (US)

(73) Assignee: VARIATION BIOTECHNOLOGIES, INC., Gatineau, QC (CA)

(21) Appl. No.: 12/999,725

(22) PCT Filed: Jun. 19, 2009
(86) PCT No.: PCT/US09/47911

§ 371 Date: Dec. 17, 2010

Related U.S. Application Data
(60) Provisional application No. 61/182,614, filed on May 29, 2009.

(51) Int. Cl. A61K 39/145; A61K 9/14; A61P 31/16

(52) U.S. Cl. 424/499; 424/186.1

(57) ABSTRACT

The present application provides compositions and methods useful for treating influenza. As described herein, the compositions and methods are based on the development of peptides and peptide combinations which exhibit immunogenic properties against influenza. In some embodiments, the peptide combinations induce a protective response against multiple strains of influenza, e.g., seasonal strains of influenza or even the new pandemic influenza A (H1N1) virus of swine origin.

### RELATED APPLICATIONS

This application claims priority to and benefit of PCT Patent Application No. PCT/US08/67471 filed Jun. 19, 2008 and U.S. Provisional Application No. 61/182,614 filed May 29, 2009. The contents of these priority applications are incorporated herein by reference in their entirety.

### BACKGROUND

Influenza is a common infectious disease of the respiratory system associated with the Orthomyxoviridae family of viruses. Because of the high degree of variability of the virus, vaccination is typically required on a yearly basis with a reformulated vaccine that takes into account strain variations. The vaccine composition developed each year in the United States is determined by the Department of Food and Drug Administration Vaccines and the Related Biologicals Advisory Committee. The World Health Organization (WHO) similarly operates a global surveillance network of laboratories, for detection of new influenza variants, e.g., see Lavanchy, Vaccine 17:S24 (1999). Selection is based on antigenic analysis of recently isolated influenza viruses, the patterns of spread of antigenic variants, and the antibody responses of recently vaccinated individuals.

Influenza A and B are the two types of influenza viruses that cause epidemic human disease. Influenza A viruses are further categorized into subtypes on the basis of two surface antigens: hemagglutinin (HA) and neuraminidase (N). Influenza B viruses are not categorized into subtypes. Since 1977, influenza A (H1N1) viruses, influenza A (H3N2) viruses and influenza B viruses have been in global circulation. Vaccination is recognized as the single most effective way of preventing or attenuating influenza for those at high risk of serious illness from influenza infection and related complications. The inoculation of antigen prepared from inactivated influenza virus stimulates the production of specific antibodies. Protection is afforded only against those strains of virus from which the vaccine is prepared or closely related strains.

Each year's vaccine contains three virus strains (usually two type A and one type B) representing the influenza viruses that are believed likely to circulate in the coming winter. The antigenic characteristics of current and emerging influenza virus strains provide the basis for selecting strains included in each year's vaccine. The WHO reviews the world epidemiological situation annually and if necessary recommends new strains based on the current epidemiological evidence.

Despite the recomposition, it is not possible for a vaccine to include all the different strains actively infecting people in the world during a particular season. In addition, a relatively long length of time is also required to formulate and prepare sufficient quantities of vaccine doses for responding to seasonal increases in flu infections. Typically, it can take over six months to prepare a vaccine. As a result, new or overlooked influenza strains can become prominent during that six month period, leading to an epidemic. In April 2009 a novel influenza A (H1N1) virus of swine origin was first detected. It is a quadruple reassortant compared to the previously described H1N1 subtype. The initial outbreak of the virus was in Mexico but the epidemic has spread rapidly over the borders through the United States and Canada and now almost all international countries are reporting new cases. There remains a need in the art for improved compositions and methods for treating influenza. In particular, there is a need for compositions that have broad immunogenicity against both seasonal influenza strains recommended by WHO and against new emerging pandemic influenza strains such as the new influenza A (H1N1) virus of swine origin.

### SUMMARY

The present application provides compositions and methods useful for treating influenza. As described herein, the compositions and methods are based on the development of peptides and peptide combinations which exhibit immunogenic properties against influenza.

In some embodiments, the peptide combinations induce a protective response against multiple strains of influenza, e.g., seasonal strains of influenza or even the new pandemic influenza A (H1N1) virus of swine origin.

In some embodiments, the compositions are administered parenterally (e.g., via intramuscular injection). In some embodiments, the parenteral compositions include a vesicle that comprises a non-ionic surfactant. In some embodiments, the parenteral compositions include TLR-4 agonist adjuvants. In some embodiments at least a portion of the TLR-4 agonist adjuvant present in the parenteral composition is physically associated with the vesicle.

While influenza vaccines are currently limited to the aforementioned parenteral administration routes (e.g., intramuscular injection), we have identified compositions that induce a protective response when administered orally. Therefore, in some embodiments, the compositions are administered orally. In some embodiments, the oral compositions include a bilosome. In some embodiments, the oral compositions include TLR-3 agonist adjuvants. In some embodiments at least a portion of the TLR-3 agonist adjuvant present in the oral composition is physically associated with the bilosome.

### BRIEF DESCRIPTION OF THE DRAWINGS

FIGS. 1A-B show the specificity of exemplary peptide compositions as detected by competition micro-neutralization assay. Human sera were diluted and incubated with selected peptide compositions and subsequently with influenza viruses. After incubation, MDCK (Madin-Darby Canine Kidney) cells were added and the plates were developed 18-22 hours later. (A) Competition micro-neutralization was detected against influenza New Calcdonia (A/NC/20/99) using human sera. (B) Competition micro-neutralization was detected against influenza Wisconsin (A/W is/67/05) using human sera.

FIG. 2 shows the cell-mediated immune responses triggered by exemplary peptide compositions as detected by IFNγ-ELISPOT assay expressed as spot forming cells (SFC) per million cells. Peripheral blood mononuclear cells (PBMCs) from an influenza-vaccinated individual were cultured in the presence of selected peptides. The plates were developed after 2 days and numbers of spots in each well were magnified and counted.

FIG. 3 shows median daily fever counts during a 10 day period after challenge with 1×10⁶ plaque-forming units (pfu) of H3N2 (A/Wisconsin/2005) virus in animals vaccinated with peptide composition INF-61P compared to non-vaccinated control animals. Fever count was defined as every minute an animal had a body temperature of 40° C. or higher; median fever counts among 4 animals per group were calculated every 3 hours. The control and vaccinated animals had comparable starting body temperatures (see Day 0).

FIG. 4 shows weight loss during a 10 day period after challenge with 1×10⁶ pfu of H3N2 (A/Wisconsin/2005) virus in animals vaccinated with peptide composition INF-61P compared to non-vaccinated control animals.

FIG. 5 shows mucosal IgA responses directed against influenza virus (FLUVIRAL) that were seen in rectal wash (top panel) and nasal wash samples (bottom panel) from ferrets immunized with peptide composition INF-61P. The data is represented as fold increase relative to pre-vaccination responses in each animal, and has been normalized for the total amount of IgA present in the different samples. Comparative data that was obtained with a commercial influenza vaccine (FLUVIRAL) are also provided.

FIG. 6 shows serum IgA responses directed against recombinant HA protein from influenza type B (Ohio) that were seen in serum from ferrets immunized with peptide composition SFV2. Responses measured by ELISA (reported as optical density) from pre-vaccination and post-vaccination serum samples for each animal are shown in paired left and right columns, respectively. Strong positive responses can be seen in animals # 4850 and # 4860.

FIG. 7 shows broadly reactive cellular (CTL) immunity induced with lipidated peptide composition TNF-09L-A. Aged (>12 months) HLA (A*0201) transgenic mice were vaccinated three times with the composition which targets a variable region in the nucleoprotein of type A influenza. The composition also included an alum adjuvant. A control group only received the adjuvant. Splenocytes were infected with divergent strains of influenza from multiple subtypes: H3N2 (A/HK/1/18, A/VICtoria/3/75) and H1N1 (A/NC/20/99, A/PR/8/34). Spots represent the frequency of IFNγ-secreting T cells specific for each of the strains of virus, and error bars represent the standard deviation among mice in each group (n=3). The results are presented from left to right as follows: H3N2 (A/HK/1/18), H3N2 (A/VICtoria/3/75), H1N1 (A/NC/20/99) and H1N1 (A/PR/8/34).

FIG. 8 shows immune responses to a peptide composition administered orally, with or without a TLR-3 agonist adjuvant (poly I:C). Mice (n=4 per group) were immunized orally 4 times (Days 0, 3, 14, and 17). Splenocytes were harvested after vaccinations and stimulated in vitro with the same peptide composition. Responses were measured using an IFNγ ELISPOT assay. As shown in FIG. 8, animals that received the adjuvant showed a much greater response than did animals that received vaccine alone.

FIG. 9 shows the sequences of SEQ ID NOs. 16, 17 and 18.

FIGS. 10A-B show serum IgG responses (measured by ELISA) directed against recombinant hemagglutinin (rHA) proteins from the following subtypes of influenza type A: (A/Solomon Island/03/06) H1N1 or (A/Wisconsin/67/05) H3N2 that were measured in ferrets immunized with peptide composition SFV2 administered intramuscularly. Post immunization serum demonstrated a significant immune response in ELISA with both rHA Solomon (A) and Wisconsin (B) for ferrets immunized with peptide composition SFV2 administered intramuscularly. No IgG response was detected against rHA from influenza type B (B/Malaysia/2506/04) (data not shown). Comparative data that was obtained with a commercial influenza vaccine (VAXIGRIP) are also provided.

FIG. 11 shows viral load from nasal wash samples of ferrets immunized with peptide composition SFV2 administered intramuscularly and then subjected to viral challenge. Animals were inoculated with immunogenic peptide composition SFV2 and challenged with 2×10⁵ pfu of H1N1 (A/Solomon Island/03/06) and viral load from nasal wash samples was measured by plaque assay at the peak of viremia (Day 2). Each symbol represents the viral load measured in an individual animal. Comparative data that was obtained with a commercial influenza vaccine (VAXIGRIP) are also provided.

FIG. 12 shows humoral immunity against a potential pandemic strain of influenza in ferrets immunized with peptide composition SFV2 administered intramuscularly. Sera was collected from animals two weeks after the vaccination (prior to viral challenge) and samples were tested for reactivity with recombinant hemagglutinin (rHA) protein from the putative pandemic swine (H1N1/California/2009) isolate by ELISA. Comparative data that was obtained with a commercial influenza vaccine (VAXIGRIP) are also provided.

FIG. 13 shows the chemical structure of the exemplary TLR-4 agonist adjuvant PHAD (phosphorylated hexaacyl disaccharide from Avanti Polar Lipids, Inc. of Alabaster, Ala.).

FIG. 14 shows the chemical structure of di[3-deoxy-D-manno-octulosonyl]-lipid A (ammonium salt) another exemplary TLR-4 agonist adjuvant (from Avanti Polar Lipids, Inc. of Alabaster, Ala.).

### DEFINITIONS

Throughout the present application, several terms are employed that are defined in the following paragraphs.

As used herein, the term “immune response” refers to a response elicited in an animal. An immune response may refer to cellular immunity, humoral immunity or may involve both. An immune response may also be limited to a part of the immune system. For example, in some embodiments, an immunogenic composition may induce an increased IFNγ response. In some embodiments, an immunogenic composition may induce a mucosal IgA response (e.g., as measured in nasal and/or rectal washes). In some embodiments, an immunogenic composition may induce a systemic IgG response (e.g., as measured in serum).

As used herein, the term “immunogenic” means capable of producing an immune response in a host animal against a non-host entity (e.g., an influenza virus). In some embodiments, this immune response forms the basis of the protective immunity elicited by a vaccine against a specific infectious organism (e.g., an influenza virus).

As used herein, the term “peptide” refers to a string of at least three amino acids linked together by peptide bonds. In general, there is no upper limit on the number of amino acids in a peptide. A peptide will generally contain only natural amino acids; however, non-natural amino acids (i.e., amino acids that do not occur in nature but that can be incorporated into a polypeptide chain) may be included. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In various embodiments, the modification(s) lead to a more stable peptide (e.g., greater half-life in vivo). Suitable modifications may include cyclization of the peptide, the incorporation of D-amino acids, etc. In various embodiments, the modification(s) lead to a more immunogenic peptide. Suitable modifications may include covalent attachment of one or more lipids (e.g., without limitation, palmitoyl, myristoyl, stearoyl, lauroyl, octanoyl, decanoyl, etc.), fusion to a carrier protein (e.g., without limitation, purified protein derivative of tuberculin (PPD), tetanus toxoid, cholera toxin and its B subunit, ovalbumin, bovine serum albumin, soybean trypsin inhibitor, muramyldipeptide and analogues thereof, a cytokine or fragment thereof, etc.), etc.

As used herein, the terms “percentage homology” refer to the percentage of sequence identity between two sequences after optimal alignment as defined in the present application. Two amino acid sequences are said to be “identical” if the sequence of amino acids in the two sequences is the same when aligned for maximum correspondence as described below. Sequence comparisons between two amino acid sequences are typically performed by comparing sequences of two optimally aligned sequences over a region or “comparison window” to identify and compare regions of sequence similarity. Optimal alignment of sequences for comparison may be conducted by the local homology algorithm of Smith and Waterman, Ad. App. Math. 2:482 (1981), by the homology alignment algorithm of Neddleman and Wunsch, J. Mol. Biol. 48:443 (1970), by the search for similarity method of Pearson and Lipman, Proc. Natl. Acad. Sci. USA 85:2444 (1988), by computerized implementation of these algorithms, or by visual inspection.

“Percentage of sequence identity” is determined by comparing two optimally aligned sequences over a comparison window, where the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. This definition of sequence identity given above is the definition that would be used by one of ordinary skill in the art. The definition by itself does not need the help of any algorithm. The algorithms are only helpful to facilitate the optimal alignments of sequences, rather than calculate sequence identity. From this definition, it follows that there is a well defined and only one value for the sequence identity between two compared sequences which value corresponds to the value obtained for the optimal alignment.

As used herein, the terms “therapeutically effective amount” refer to the amount sufficient to show a meaningful benefit in a subject being treated. The therapeutically effective amount of an immunogenic composition may vary depending on such factors as the desired biological endpoint, the nature of the composition, the route of administration, the health, size and/or age of the subject being treated, etc.

As used herein, the term “treat” (or “treating”, “treated”, “treatment”, etc.) refers to the administration of an immunogenic composition to a subject who has influenza, a symptom of influenza or a predisposition toward influenza, with the purpose to alleviate, relieve, alter, ameliorate, improve or affect the influenza, a symptom or symptoms of influenza, or the predisposition toward influenza. In some embodiments, the term “treating” refers to the vaccination of a subject.

### DETAILED DESCRIPTION OF SOME EMBODIMENTS

The present application provides compositions and methods useful for treating influenza. As described herein, the compositions and methods are based on the development of peptides and peptide combinations which exhibit immunogenic properties against influenza.

In some embodiments, the peptide combinations induce a protective response against multiple strains of influenza, e.g., seasonal strains of influenza or even the new pandemic influenza A (H1N1) virus of swine origin.

In some embodiments, the compositions are administered parenterally (e.g., via intramuscular injection). In some embodiments, the compositions include TLR-4 agonist adjuvants. In some embodiments, the compositions include a vesicle that comprises a non-ionic surfactant. In some embodiments at least a portion of the TLR-4 agonist adjuvant present in the composition is physically associated with the vesicle.

While influenza vaccines are currently limited to the aforementioned parenteral administration routes (e.g., intramuscular injection), we have identified compositions that induce a protective response when administered orally. Therefore, in some embodiments, the compositions are administered orally. In some embodiments, the compositions include TLR-3 agonist adjuvants. In some embodiments, the compositions include a bilosome. In some embodiments at least a portion of the TLR-3 agonist adjuvant present in the composition is physically associated with the bilosome.

### I. Peptides

In one aspect, the present application provides peptides that can be used alone or in combination to produce an immunogenic composition for treating influenza. It is to be understood that any of these peptides may be included in an immunogenic composition and that the present application encompasses compositions that include any permutation or combination of these peptides. Section II below describes some exemplary peptide combinations.

### Type A Influenza Hemagglutinin (HA) Subtype 1 (H1) Peptides

Tables 2-6 in the Examples describe the amino acid sequences of several peptides that have been derived from type A influenza hemagglutinin (HA) subtype 1 (H1) proteins. As shown in Tables 2-5 and the Sequence Listing, some of the peptides are variants of a common consensus sequence.

In some embodiments, the present application provides peptides that comprise at least 20 contiguous amino acids of SEQ ID NO. 2 (see Table 2). It will be appreciated that any 20 contiguous amino acids of any one of the variant sequences described by the consensus sequence of SEQ ID NO. 2 is encompassed. In some embodiments, a peptide may comprise at least 21, 22, 23, 24, 25 or 26 contiguous amino acids of SEQ ID NO. 2. The present application also provides immunogenic compositions which comprise two or more of these peptides. Thus, in some embodiments, an immunogenic composition may comprise 2, 3, 4, 5, 6, 7 or 8 different peptides which comprise at least 20 contiguous amino acids of SEQ ID NO. 2. In some embodiments, an immunogenic composition may comprise 2ⁿ different peptides each comprising a different amino acid sequence of SEQ ID NO. 2, where n=1-3. In some embodiments, n=1. In some embodiments, n=2. In some embodiments, n=3.

In some embodiments, the present application provides peptides that comprise at least 20 contiguous amino acids of SEQ ID NO. 3 (see Table 3). It will be appreciated that any 20 contiguous amino acids of any one of the variant sequences described by the consensus sequence of SEQ ID NO. 3 is encompassed. In some embodiments, a peptide may comprise at least 21, 22, 23, 24, 25, 26, 27, 28, 29, 30, 31, 32 or 33 contiguous amino acids of SEQ ID NO. 3. The present application also provides immunogenic compositions which comprise two or more of these peptides. Thus, in some embodiments, an immunogenic composition may comprise 2, 3, 4, 5, 6, 7 or 8 different peptides which comprise at least 20 contiguous amino acids of SEQ ID NO. 3. In some embodiments, an immunogenic composition may comprise 2ⁿ different peptides each comprising a different amino acid sequence of SEQ ID NO. 3, where n=1-3. In some embodiments, n=1. In some embodiments, n=2. In some embodiments, n=3.

In some embodiments, the present application provides peptides that comprise at least 20 contiguous amino acids of SEQ ID NO. 4 (see Table 4). It will be appreciated that any 20 contiguous amino acids of any one of the variant sequences described by the consensus sequence of SEQ ID NO. 4 is encompassed. In some embodiments, a peptide may comprise at least 21, 22, 23, 24, 25, 26, 27, 28, 29 or 30 contiguous amino acids of SEQ ID NO. 4. The present application also provides immunogenic compositions which comprise two or more of these peptides. Thus, in some embodiments, an immunogenic composition may comprise 2, 3 or 4 different peptides which comprise at least 20 contiguous amino acids of SEQ ID NO. 4. In some embodiments, an immunogenic composition may comprise 2ⁿ different peptides each comprising a different amino acid sequence of SEQ ID NO. 4, where n=1-2. In some embodiments, n=1. In some embodiments, n=2.

In some embodiments, the present application provides peptides that comprise at least 20 contiguous amino acids of SEQ ID NO. 5 (see Table 5). It will be appreciated that any 20 contiguous amino acids of any one of the variant sequences described by the consensus sequence of SEQ ID NO. 5 is encompassed. In some embodiments, a peptide may comprise at least 21, 22, 23, 24, 25, 26, 27, 28 or 29 contiguous amino acids of SEQ ID NO. 5. The present application also provides immunogenic compositions which comprise two or more of these peptides. Thus, in some embodiments, an immunogenic composition may comprise 2, 3, 4, 5, 6, 7 or 8 different peptides which comprise at least 20 contiguous amino acids of SEQ ID NO. 5. In some embodiments, an immunogenic composition may comprise 2ⁿ different peptides each comprising a different amino acid sequence of SEQ ID NO. 5, where n=1-3. In some embodiments, n=1. In some embodiments, n=2. In some embodiments, n=3.

In some embodiments, the present application provides peptides which comprise a region having at least 80% homology with at least 40 contiguous amino acids of SEQ ID NO. 6 (see Table 6). In some embodiments, the homology may be at least 85%, 90%, 95% or 99%.

In some embodiments, the present application provides peptides that comprise at least 40 contiguous amino acids of SEQ ID NO. 6. In some embodiments, the present application provides peptides that comprise at least 50, 60, 70 or 80 contiguous amino acids of SEQ ID NO. 6. Thus, a peptide may comprise amino acids 1-81 of SEQ ID NO. 6. A peptide may also comprise amino acids 82-88 of SEQ ID NO. 6. In some embodiments, a peptide may comprise the entire sequence of SEQ ID NO. 6. In other embodiments, a peptide may consist essentially of SEQ ID NO. 6. In yet other embodiments, a peptide may consist of SEQ ID NO. 6.

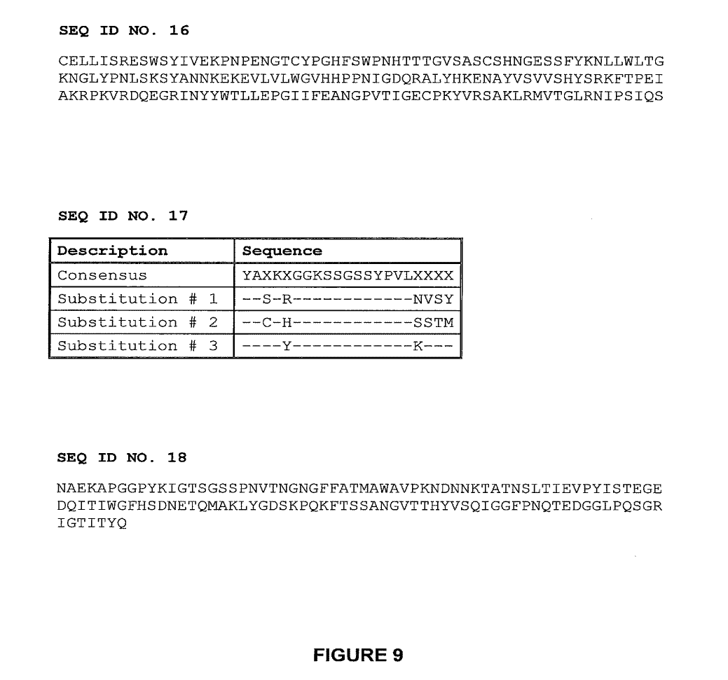

In addition to the foregoing, the present application also provides a genus of peptides that comprise a region having at least 80% homology with 20-100 contiguous amino acids of SEQ ID NO. 16 (see FIG. 9), wherein the peptide includes fewer than 100 contiguous amino acids from a type A influenza hemagglutinin (HA) protein. This genus encompasses the other H1 peptides described above. In some embodiments, the peptide may comprise a region having at least 85%, 90%, 95% or 99% homology with 20-100 contiguous amino acids of SEQ ID NO. 16. In some embodiments, the peptide may comprise a region having at least 80% homology with 20-50 contiguous amino acids of SEQ ID NO. 16. In some embodiments, the peptide may comprise a region having at least 80% homology with 50-100 contiguous amino acids of SEQ ID NO. 16.

In some embodiments, any of the aforementioned H1 peptides may comprise fewer than 30 amino acids. However, as discussed herein, in some embodiments, a peptide comprising any one of the defined regions may also be comprised within a larger peptide.

### Type A Influenza Hemagglutinin (HA) Subtype 3 (H3) Peptides

Tables 9-11 in the Examples describe the amino acid sequences of several peptides that have been derived from type A influenza hemagglutinin (HA) subtype 3 (H3) proteins. As shown in Tables 9-11 and the Sequence Listing, these peptides are all variants of the consensus sequence of SEQ ID NO. 17 (see FIG. 9).

In some embodiments, the present application provides peptides that comprise a sequence of SEQ ID NO. 17. It will be appreciated that any one of the variant sequences described by the consensus sequence of SEQ ID NO. 17 is encompassed.

In some embodiments, the present application provides peptides that comprise a sequence of SEQ ID NO. 9 (see Table 9). It will be appreciated that any one of the variant sequences described by the consensus sequence of SEQ ID NO. 9 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 9. In some embodiments, an immunogenic composition may comprise 2ⁿ different peptides each comprising a different amino acid sequence of SEQ ID NO. 9, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 9.

In some embodiments, the present application provides peptides that comprise a sequence of SEQ ID NO. 10 (see Table 10). It will be appreciated that any one of the variant sequences described by the consensus sequence of SEQ ID NO. 10 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 10. In some embodiments, an immunogenic composition may comprise 2ⁿ different peptides each comprising a different amino acid sequence of SEQ ID NO. 10, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 10.

In some embodiments, the present application provides peptides that comprise a sequence of SEQ ID NO. 11 (see Table 11). It will be appreciated that any one of the variant sequences described by the consensus sequence of SEQ ID NO. 11 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 11. In some embodiments, an immunogenic composition may comprise 2ⁿ different peptides each comprising a different amino acid sequence of SEQ ID NO. 11, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 11.

In some embodiments, any of the aforementioned H3 peptides may comprise fewer than 30 amino acids. However, as discussed herein, in some embodiments, a peptide comprising any one of the defined regions may also be comprised within a larger peptide.

Table 7 in the Examples describes the amino acid sequences of another peptide that has been derived from type A influenza hemagglutinin (HA) subtype 3 (H3) proteins. The present application provides a peptide comprising a region having at least 80% homology with at least 20 contiguous amino acids of SEQ ID NO. 7, wherein the peptide includes fewer than 100 contiguous amino acids from a type A influenza hemagglutinin protein. In some embodiments, a peptide may comprise a region having at least 85%, 90%, 95% or 99% homology with at least 20 contiguous amino acids of SEQ TD NO. 7. In some embodiments, a peptide may comprise a region having at least 80% homology with at least 50, 60, 70 or 80 contiguous amino acids of SEQ ID NO. 7. In some embodiments, a peptide may comprise the entire sequence of SEQ ID NO. 7. In other embodiments, a peptide may consist essentially of SEQ ID NO. 7. In yet other embodiments, a peptide may consist of SEQ ID NO. 7. In some embodiments, the peptide may comprise fewer than 100 amino acids, e.g., fewer than 90, 80, 70, 60, 50 or 40 amino acids. However, as discussed herein, in some embodiments, a peptide comprising any one of the defined regions may also be comprised within a larger peptide.

### Type B Influenza Hemagglutinin (HA) Peptides

Tables 8 and 12 in the Examples describe the amino acid sequences of several peptides that have been derived from type B influenza hemagglutinin (HA) proteins.

In some embodiments, the present application provides peptides that comprise a region having at least 80% homology with at least 20 contiguous amino acids from positions 2-49 or 68-121 of SEQ ID NO. 18 (see FIG. 9), wherein the peptide includes fewer than 100 contiguous amino acids from a type B influenza hemagglutinin protein. In some embodiments, a peptide may comprise a region having at least 85%, 90%, 95% or 99% homology with at least 20 contiguous amino acids from positions 2-49 or 68-121 of SEQ ID NO. 18. In some embodiments, a peptide may comprise a region having at least 80% homology with at least 25, 30, 35, 40 or 45 contiguous amino acids from positions 2-49 or 68-121 of SEQ ID NO. 18.

In some embodiments, a peptide may comprise a region having at least 80% homology with the entire sequence of SEQ ID NO. 12 (see Table 12). In some embodiments, the level of homology may be at least 85%, 90%, 95% or 99%. In some embodiments, a peptide may comprise the entire sequence of SEQ ID NO. 12. In some embodiments, a peptide may consist essentially of SEQ ID NO. 12. In some embodiments, a peptide may consist of SEQ ID NO. 12.

In some embodiments, a peptide may comprise a region having at least 80% homology with the entire sequence of SEQ ID NO. 13 (see Table 12). In some embodiments, the level of homology may be at least 85%, 90%, 95% or 99%. In some embodiments, a peptide may comprise the entire sequence of SEQ ID NO. 13. In some embodiments, a peptide may consist essentially of SEQ ID NO. 13. In some embodiments, a peptide may consist of SEQ ID NO. 13.

In some embodiments, a peptide may comprise a first region having at least 80% homology with at least 20 contiguous amino acids from positions 2-49 and a second region having at least 80% homology with at least 20 contiguous amino acids from positions 68-121 of SEQ ID NO. 18. In some embodiments, the homology may be higher, e.g., at least 85%, 90%, 95% or 99%. In some embodiments, the homology in the first and/or second region may span at least 25, 20, 35, 40 or 45 contiguous amino acids from positions 2-49 or 68-121 of SEQ ID NO. 18.

In some embodiments, a peptide may comprise a region having at least 80% homology with the entire sequence of SEQ ID NO. 8 (see Table 8). In some embodiments, the level of homology may be at least 85%, 90%, 95% or 99%. In some embodiments, a peptide may comprise the entire sequence of SEQ ID NO. 8. In other embodiments, a peptide may consist essentially of or consist of the sequence of SEQ ID NO. 8.

In some embodiments, any of the aforementioned Type B HA peptides may comprise fewer than 100 amino acids, e.g., fewer than 90, 80, 70, 60, 50 or 40 amino acids. However, as discussed herein, in some embodiments, a peptide comprising any one of the defined regions may also be comprised within a larger peptide.

### Type A Influenza Nucleoprotein (NP) Peptides

Tables 13 and 14 in the Examples describe the amino acid sequences of several peptides that have been derived from type A influenza nucleoproteins (NP). The peptides in Table 13 are variants of the consensus sequence of SEQ ID NO. 14. The peptides in Table 14 are variants of the consensus sequence of SEQ ID NO. 15.

In some embodiments, the present application provides peptides that comprise a sequence of SEQ ID NO. 14 (see Table 13), wherein the peptide includes fewer than 20 contiguous amino acids from a type A influenza nucleoprotein. It will be appreciated that any one of the variant sequences described by the consensus sequence of SEQ ID NO. 14 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 14. In some embodiments, an immunogenic composition may comprise 2ⁿ different peptides each comprising a different amino acid sequence of SEQ ID NO. 14, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 14.

In some embodiments, the present application provides peptides that comprise a sequence of SEQ ID NO. 15 (see Table 14), wherein the peptide includes fewer than 20 contiguous amino acids from a type A influenza nucleoprotein. It will be appreciated that any one of the variant sequences described by the consensus sequence of SEQ ID NO. 15 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 15. In some embodiments, an immunogenic composition may comprise 2ⁿ different peptides each comprising a different amino acid sequence of SEQ ID NO. 15, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 15.

In some embodiments, any of the aforementioned NP peptides may comprise fewer than 30 amino acids. However, as discussed herein, in some embodiments, a peptide comprising any one of the defined regions may also be comprised within a larger peptide.

### Other Influenza Peptides

The present application provides other peptides which include sequences from more than one influenza protein. Thus, in some embodiments, the present application provides peptides which comprise an amino acid sequence of SEQ ID NO. 1 (see Table 1). As shown in Table 1 and the Sequence Listing, SEQ ID NO. 1 is a consensus sequence. The consensus sequence was derived from sequences in Type B influenza HA proteins and Type A influenza HA H3 proteins. It will be appreciated that any one of the variant sequences described by the consensus sequence of SEQ ID NO. 1 is encompassed. The present application also provides immunogenic compositions which comprise two or more of these peptides. Thus, in some embodiments, an immunogenic composition may comprise 2, 3, 4, 5, 6, 7, 8, 9, 10, 11, 12, 13, 14, 15 or 16 different peptides that comprise a sequence of SEQ ID NO. 1. In some embodiments, an immunogenic composition may comprise 2ⁿ different peptides each comprising a different amino acid sequence of SEQ ID NO. 1, where n=1-4. In some embodiments, n=1. In some embodiments, n=2. In some embodiments, n=3. In some embodiments, n=4. It is to be understood that the present application also provides peptides which consist essentially of or consist of an amino acid sequence of SEQ ID NO. 1. It is also to be understood that in some embodiments a peptide may comprise fewer than 30 amino acids. However, as discussed herein, in some embodiments, a peptide comprising any one of the defined regions may also be comprised within a larger peptide.

### II. Peptide Combinations

In one aspect, the present application provides immunogenic compositions that include combinations of peptides described in Section I. It is to be understood that the following exemplary combinations are non-limiting and that the present application encompasses all permutations and combinations of the peptides described in Section I. It is also to be understood that other peptides (including traditional influenza protein antigens found in existing influenza vaccines) may be added to any of the immunogenic compositions described herein. In some embodiments, each peptide in an immunogenic composition is independently immunogenic.

In some embodiments, at least one peptide comprises a region with 20 or more contiguous amino acids from a non-influenza protein. In some embodiments each peptide comprises a region with 20 or more contiguous amino acids from a non-influenza protein. In some embodiments, at least two peptides of the composition are present within a single protein.

In some embodiments, the present application provides immunogenic compositions that include one or more Type A influenza HA H1 peptides and one or more Type A influenza HA H3 peptides from Section I.

In some embodiments, the present application provides immunogenic compositions that include one or more Type A influenza HA H1 peptides and one or more Type B influenza HA peptides from Section I.

In some embodiments, the present application provides immunogenic compositions that include one or more Type A influenza HA H3 peptides and one or more Type B influenza HA peptides from Section I.

In some embodiments, the present application provides immunogenic compositions that include one or more Type A influenza HA H1 peptides, one or more Type A influenza HA H3 peptides and one or more Type B influenza HA peptides from Section I.

In some embodiments, one or more Type A influenza NP peptides from Section 1 can be included in any one of the aforementioned combinations.

In some embodiments, the present application provides immunogenic compositions which comprise a first peptide comprising a region having at least 80% homology with 20-100 contiguous amino acids of SEQ ID NO. 16, wherein the first peptide includes fewer than 100 contiguous amino acids from a type A influenza hemagglutinin protein; a second peptide comprising an amino acid sequence of SEQ ID NO. 17; and a third peptide comprising a region having at least 80% homology with at least 20 contiguous amino acids from positions 2-49 or 68-121 of SEQ ID NO. 18, wherein the third peptide includes fewer than 100 contiguous amino acids from a type B influenza hemagglutinin protein. In some embodiments, the first and third peptides comprise fewer than 100 amino acids and the second peptide comprises fewer than 30 amino acids.

In some embodiments, a fourth peptide comprising an amino acid sequence of SEQ ID NO. 14 or 15 is also included, wherein the fourth peptide includes fewer than 20 contiguous amino acids from a type A influenza nucleoprotein.

### Exemplary First Peptides (Type A Influenza HA H1)

In some embodiments, the first peptide may comprise a region having at least 85%, 90%, 95% or 99% homology with 20-100 contiguous amino acids of SEQ ID NO. 16. In some embodiments, the first peptide may comprise a region having at least 80% homology with 20-50 contiguous amino acids of SEQ ID NO. 16. In some embodiments, the first peptide may comprise a region having at least 80% homology with 50-100 contiguous amino acids of SEQ ID NO. 16.

In some embodiments, the first peptide may comprise at least 20 contiguous amino acids of SEQ ID NO. 2 (see Table 2). It will be appreciated that any 20 contiguous amino acids of any one of the variant sequences described by the consensus sequence of SEQ ID NO. 2 is encompassed. In some embodiments, a first peptide may comprise at least 21, 22, 23, 24, 25 or 26 contiguous amino acids of SEQ ID NO. 2. The present application also provides immunogenic compositions which comprise two or more of these first peptides. Thus, in some embodiments, an immunogenic composition may comprise 2, 3, 4, 5, 6, 7 or 8 different first peptides which comprise at least 20 contiguous amino acids of SEQ ID NO. 2. In some embodiments, an immunogenic composition may comprise 2ⁿ different first peptides each comprising a different amino acid sequence of SEQ ID NO. 2, where n=1-3. In some embodiments, n=1. In some embodiments, n=2. In some embodiments, n=3.

In some embodiments, the first peptide may comprise at least 20 contiguous amino acids of SEQ ID NO. 3 (see Table 3). It will be appreciated that any 20 contiguous amino acids of any one of the variant sequences described by the consensus sequence of SEQ ID NO. 3 is encompassed. In some embodiments, a first peptide may comprise at least 21, 22, 23, 24, 25, 26, 27, 28, 29, 30, 31, 32 or 33 contiguous amino acids of SEQ ID NO. 3. The present application also provides immunogenic compositions which comprise two or more of these first peptides. Thus, in some embodiments, an immunogenic composition may comprise 2, 3, 4, 5, 6, 7 or 8 different first peptides which comprise at least 20 contiguous amino acids of SEQ ID NO. 3. In some embodiments, an immunogenic composition may comprise 2ⁿ different first peptides each comprising a different amino acid sequence of SEQ ID NO. 3, where n=1-3. In some embodiments, n=1. In some embodiments, n=2. In some embodiments, n=3.

In some embodiments, the first peptide may comprise at least 20 contiguous amino acids of SEQ ID NO. 4 (see Table 4). It will be appreciated that any 20 contiguous amino acids of any one of the variant sequences described by the consensus sequence of SEQ ID NO. 4 is encompassed. In some embodiments, a first peptide may comprise at least 21, 22, 23, 24, 25, 26, 27, 28, 29 or 30 contiguous amino acids of SEQ ID NO. 4. The present application also provides immunogenic compositions which comprise two or more of these first peptides. Thus, in some embodiments, an immunogenic composition may comprise 2, 3 or 4 different first peptides which comprise at least 20 contiguous amino acids of SEQ ID NO. 4. In some embodiments, an immunogenic composition may comprise 2ⁿ different first peptides each comprising a different amino acid sequence of SEQ ID NO. 4, where n=1-2. In some embodiments, n=1. In some embodiments, n=2.

In some embodiments, the first peptide may comprise at least 20 contiguous amino acids of SEQ ID NO. 5 (see Table 5). It will be appreciated that any 20 contiguous amino acids of any one of the variant sequences described by the consensus sequence of SEQ ID NO. 5 is encompassed. In some embodiments, a first peptide may comprise at least 21, 22, 23, 24, 25, 26, 27, 28 or 29 contiguous amino acids of SEQ ID NO. 5. The present application also provides immunogenic compositions which comprise two or more of these first peptides. Thus, in some embodiments, an immunogenic composition may comprise 2, 3, 4, 5, 6, 7 or 8 different first peptides which comprise at least 20 contiguous amino acids of SEQ ID NO. 5. In some embodiments, an immunogenic composition may comprise 2ⁿ different first peptides each comprising a different amino acid sequence of SEQ ID NO. 5, where n=1-3. In some embodiments, n=1. In some embodiments, n=2. In some embodiments, n=3.

In some embodiments, the first peptide may comprise a region having at least 80% homology with at least 40 contiguous amino acids of SEQ ID NO. 6 (see Table 6). In some embodiments, the homology may be at least 85%, 90%, 95% or 99%. In some embodiments, the first peptide may comprise at least 40 contiguous amino acids of SEQ ID NO. 6. In some embodiments, the first peptide may comprise at least 50, 60, 70 or 80 contiguous amino acids of SEQ ID NO. 6. Thus, a first peptide may comprise amino acids 1-81 of SEQ ID NO. 6. A first peptide may also comprise amino acids 82-88 of SEQ ID NO. 6. In some embodiments, a first peptide may comprise the entire sequence of SEQ ID NO. 6. In other embodiments, a first peptide may consist essentially of SEQ ID NO. 6. In yet other embodiments, a first peptide may consist of SEQ ID NO. 6.

In some embodiments, any of the aforementioned first peptides may comprise fewer than 30 amino acids. However, as discussed herein, in some embodiments, a first peptide comprising any one of the defined regions may also be comprised within a larger peptide.

### Exemplary Second Peptides (Type A Influenza HA H3)

In some embodiments, the second peptide may comprise a sequence of SEQ ID NO. 17. It will be appreciated that any one of the variant sequences described by the consensus sequence of SEQ ID NO. 17 is encompassed.

In some embodiments, the second peptide may comprise a sequence of SEQ ID NO. 9 (see Table 9). It will be appreciated that any one of the variant sequences described by the consensus sequence of SEQ ID NO. 9 is encompassed. The present application also provides immunogenic compositions which comprise two or more of these second peptides. Thus, in some embodiments, an immunogenic composition may comprise 2, 3, 4, 5, 6, 7, 8, 9, 10, 11, 12, 13, 14, 15 or 16 different second peptides that comprise a sequence of SEQ ID NO. 9. In some embodiments, an immunogenic composition may comprise 2ⁿ different second peptides each comprising a different amino acid sequence of SEQ ID NO. 9, where n=1-4. In some embodiments, n=1. In some embodiments, n=2. In some embodiments, n=3. In some embodiments, n=4. It is to be understood that the present application also provides second peptides which consist essentially of or consist of an amino acid sequence of SEQ ID NO. 9.

In some embodiments, the second peptide may comprise a sequence of SEQ ID NO. 10 (see Table 10). It will be appreciated that any one of the variant sequences described by the consensus sequence of SEQ ID NO. 10 is encompassed. The present application also provides immunogenic compositions which comprise two or more of these second peptides. Thus, in some embodiments, an immunogenic composition may comprise 2, 3, 4, 5, 6, 7, 8, 9, 10, 11, 12, 13, 14, 15 or 16 different second peptides that comprise a sequence of SEQ ID NO. 10. In some embodiments, an immunogenic composition may comprise 2ⁿ different second peptides each comprising a different amino acid sequence of SEQ ID NO. 10, where n=1-4. In some embodiments, n=1. In some embodiments, n=2. In some embodiments, n=3. In some embodiments, n=4. It is to be understood that the present application also provides second peptides which consist essentially of or consist of an amino acid sequence of SEQ ID NO. 10.

In some embodiments, the second peptide may comprise a sequence of SEQ ID NO. 11 (see Table 11). It will be appreciated that any one of the variant sequences described by the consensus sequence of SEQ ID NO. 11 is encompassed. The present application also provides immunogenic compositions which comprise two or more of these second peptides. Thus, in some embodiments, an immunogenic composition may comprise 2, 3, 4, 5, 6, 7, 8, 9, 10, 11, 12, 13, 14, 15 or 16 different second peptides that comprise a sequence of SEQ ID NO. 11. In some embodiments, an immunogenic composition may comprise 2ⁿ different second peptides each comprising a different amino acid sequence of SEQ ID NO. 11, where n=1-4. In some embodiments, n=1. In some embodiments, n=2. In some embodiments, n=3. In some embodiments, n=4. It is to be understood that the present application also provides second peptides which consist essentially of or consist of an amino acid sequence of SEQ ID NO. 11.

In some embodiments, any of the aforementioned second peptides may comprise fewer than 30 amino acids. However, as discussed herein, in some embodiments, a second peptide comprising any one of the defined regions may also be comprised within a larger peptide.

### Exemplary Third Peptides (Type B Influenza HA)

In some embodiments, a third peptide may comprise a region having at least 85%, 90%, 95% or 99% homology with at least 20 contiguous amino acids from positions 2-49 or 68-121 of SEQ ID NO. 18. In some embodiments, a third peptide may comprise a region having at least 80% homology with at least 25, 30, 35, 40 or 45 contiguous amino acids from positions 2-49 or 68-121 of SEQ ID NO. 18.

In some embodiments, a third peptide may comprise a region having at least 80% homology with the entire sequence of SEQ ID NO. 12 (see Table 12). In some embodiments, the level of homology may be at least 85%, 90%, 95% or 99%. In some embodiments, a third peptide may comprise the entire sequence of SEQ ID NO. 12. In some embodiments, a third peptide may consist essentially of SEQ ID NO. 12. In some embodiments, a third peptide may consist of SEQ ID NO. 12.

In some embodiments, a third peptide may comprise a region having at least 80% homology with the entire sequence of SEQ ID NO. 13 (see Table 12). In some embodiments, the level of homology may be at least 85%, 90%, 95% or 99%. In some embodiments, a third peptide may comprise the entire sequence of SEQ ID NO. 13. In some embodiments, a third peptide may consist essentially of SEQ ID NO. 13. In some embodiments, a third peptide may consist of SEQ ID NO. 13.

In some embodiments, a third peptide may comprise a first region having at least 80% homology with at least 20 contiguous amino acids from positions 2-49 and a second region having at least 80% homology with at least 20 contiguous amino acids from positions 68-121 of SEQ ID NO. 18. In some embodiments, the homology may be higher, e.g., at least 85%, 90%, 95% or 99%. In some embodiments, the homology in the first and/or second region may span at least 25, 20, 35, 40 or 45 contiguous amino acids from positions 2-49 or 68-121 of SEQ ID NO. 18.

In some embodiments, a third peptide may comprise a region having at least 80% homology with the entire sequence of SEQ ID NO. 8. In some embodiments, the level of homology may be at least 85%, 90%, 95% or 99%. In some embodiments, a third peptide may comprise the entire sequence of SEQ ID NO. 8. In other embodiments, a third peptide may consist essentially of or consist of the sequence of SEQ ID NO. 8.

In some embodiments, an immunogenic composition may include two different third peptides one of which comprises a region having at least 80% homology with at least 20 contiguous amino acids from positions 2-49 of SEQ ID NO. 18 while the other comprises a region having at least 80% homology with at least 20 contiguous amino acids from positions 68-121 of SEQ ID NO. 18. In some embodiments, the level of homology may be at least 85%, 90%, 95% or 99% in one or both of the peptides.

In some embodiments, any of the aforementioned third peptides may comprise fewer than 100 amino acids, e.g., fewer than 90, 80, 70, 60, 50 or 40 amino acids. However, as discussed herein, in some embodiments, a third peptide comprising any one of the defined regions may also be comprised within a larger peptide.

### Exemplary Fourth Peptides (Type A Influenza NP)

In some embodiments, an optional fourth peptide may comprise a sequence of SEQ ID NO. 14 (see Table 13). It will be appreciated that any one of the variant sequences described by the consensus sequence of SEQ ID NO. 14 is encompassed. The present application also provides immunogenic compositions which comprise two or more of these fourth peptides. Thus, in some embodiments, an immunogenic composition may comprise 2, 3, 4, 5, 6, 7, 8, 9, 10, 11, 12, 13, 14, 15 or 16 different fourth peptides that comprise a sequence of SEQ ID NO. 14. In some embodiments, an immunogenic composition may comprise 2ⁿ different fourth peptides each comprising a different amino acid sequence of SEQ ID NO. 14, where n=1-4. In some embodiments, n=1. In some embodiments, n=2. In some embodiments, n=3. In some embodiments, n=4. It is to be understood that the immunogenic composition may also include fourth peptides which consist essentially of or consist of an amino acid sequence of SEQ ID NO. 14.

In some embodiments, an optional fourth peptide may comprise a sequence of SEQ ID NO. 15 (see Table 14). It will be appreciated that any one of the variant sequences described by the consensus sequence of SEQ ID NO. 15 is encompassed. The present application also provides immunogenic compositions which comprise two or more of these fourth peptides. Thus, in some embodiments, an immunogenic composition may comprise 2, 3, 4, 5, 6, 7, 8, 9, 10, 11, 12, 13, 14, 15 or 16 different fourth peptides that comprise a sequence of SEQ ID NO. 15. In some embodiments, an immunogenic composition may comprise 2ⁿ different fourth peptides each comprising a different amino acid sequence of SEQ ID NO. 15, where n=1-4. In some embodiments, n=1. In some embodiments, n=2. In some embodiments, n=3. In some embodiments, n=4. It is to be understood that the immunogenic composition may also include fourth peptides which consist essentially of or consist of an amino acid sequence of SEQ ID NO. 15.

In some embodiments, any of the aforementioned fourth peptides may comprise fewer than 30 amino acids. However, as discussed herein, in some embodiments, a fourth peptide comprising any one of the defined regions may also be comprised within a larger peptide.

### Exemplary Combinations of Peptides

In some embodiments, an immunogenic composition may include a first peptide which comprises a region having at least 80% homology with SEQ ID NO. 6, a third peptide which comprises a region having at least 80% homology with SEQ ID NO. 8 and 2ⁿ different second peptides each comprising a different amino acid sequence of SEQ ID NO. 9, 10 or 11, where n=1-4. In one embodiment the second peptides each comprise a different amino acid sequence of SEQ ID NO. 11. In one embodiment n=4. In one embodiment, the composition further comprises 2^{m} different fourth peptides each comprising a different amino acid sequence of SEQ ID NO. 14, where m=1-4. In one embodiment, the composition further comprises 2^{m} different fourth peptides each comprising a different amino acid sequence of SEQ ID NO. 15, where m=1-4. In one embodiment m=4.

In some embodiments, an immunogenic composition may include a first peptide which comprises a region having at least 90% homology with SEQ ID NO. 6, a third peptide which comprises a region having at least 90% homology with SEQ ID NO. 8 and 2ⁿ different second peptides each comprising a different amino acid sequence of SEQ ID NO. 9, 10 or 11, where n=1-4. In one embodiment the second peptides each comprise a different amino acid sequence of SEQ ID NO. 11. In one embodiment n=4. In one embodiment, the composition further comprises 2^{m} different fourth peptides each comprising a different amino acid sequence of SEQ ID NO. 14, where m=1-4. In one embodiment, the composition further comprises 2^{m} different fourth peptides each comprising a different amino acid sequence of SEQ ID NO. 15, where m=1-4. In one embodiment m=4.

In some embodiments, an immunogenic composition may include a first peptide which comprises a region having at least 95% homology with SEQ ID NO. 6, a third peptide which comprises a region having at least 95% homology with SEQ ID NO. 8 and 2ⁿ different second peptides each comprising a different amino acid sequence of SEQ ID NO. 9, 10 or 11, where n=1-4. In one embodiment the second peptides each comprise a different amino acid sequence of SEQ ID NO. 11. In one embodiment n=4. In one embodiment, the composition further comprises 2^{m} different fourth peptides each comprising a different amino acid sequence of SEQ ID NO. 14, where m=1-4. In one embodiment, the composition further comprises 2^{m} different fourth peptides each comprising a different amino acid sequence of SEQ ID NO. 15, where m=1-4. In one embodiment m=4.

In some embodiments, an immunogenic composition may include a first peptide which comprises SEQ ID NO. 6, a third peptide which comprises SEQ ID NO. 8 and 2ⁿ different second peptides each comprising a different amino acid sequence of SEQ ID NO. 9, 10 or 11, where n=1-4. In one embodiment the second peptides each comprise a different amino acid sequence of SEQ ID NO. 11. In one embodiment n=4. In one embodiment, the composition further comprises 2^{m} different fourth peptides each comprising a different amino acid sequence of SEQ ID NO. 14, where m=1-4. In one embodiment, the composition further comprises 2^{m} different fourth peptides each comprising a different amino acid sequence of SEQ ID NO. 15, where m=1-4. In one embodiment m=4.

In some embodiments, an immunogenic composition may include a first peptide which consists essentially of SEQ ID NO. 6, a third peptide which consists essentially of SEQ ID NO. 8 and 2ⁿ different second peptides each consisting essentially of a different amino acid sequence of SEQ TD NO. 9, 10 or 11, where n=1-4. In one embodiment the second peptides each consist essentially of a different amino acid sequence of SEQ ID NO. 11. In one embodiment n=4. In one embodiment, the composition further comprises 2^{m} different fourth peptides each consisting essentially of a different amino acid sequence of SEQ ID NO. 14, where m=1-4. In one embodiment, the composition further comprises 2^{m} different fourth peptides each consisting essentially of a different amino acid sequence of SEQ ID NO. 15, where m=1-4. In one embodiment m=4.

In some embodiments, an immunogenic composition may include a first peptide which consists of SEQ ID NO. 6, a third peptide which consists of SEQ ID NO. 8 and 2ⁿ different second peptides each consisting of a different amino acid sequence of SEQ ID NO. 9, 10 or 11, where n=1-4. In one embodiment the second peptides each consist of a different amino acid sequence of SEQ ID NO. 11. In one embodiment n=4. In one embodiment, the composition further comprises 2^{m} different fourth peptides each consisting of a different amino acid sequence of SEQ ID NO. 14, where m=1-4. In one embodiment, the composition further comprises 2^{m} different fourth peptides each consisting of a different amino acid sequence of SEQ ID NO. 15, where m=1-4. In one embodiment m=4.

### III. Peptide Synthesis

Peptides that are described herein may be synthesized using any known method in the art (including recombinant methods). In various embodiments, peptides may be synthesized by solid phase peptide synthesis (SPPS). In SPPS, the C-terminal amino acid is attached to a solid phase (typically a cross-linked resin such as a polystyrene or polyethylene glycol-containing resin) via an acid labile bond with a linker molecule. The solid phase used is generally insoluble in the solvents used for synthesis, making it relatively simple and fast to wash away excess reagents and by-products. The N-terminus is protected with a protecting group (e.g., an Fmoc group) which is stable in acid, but removable by base. Side chain functional groups are protected with base stable, acid labile groups. The SPSS technique then involves incorporating N-α-protected amino acids into the growing peptide chain while the C-terminus remains attached to the solid phase. Example 1 describes an exemplary SPSS process

In general, this process can be automated and commercially available equipment can be used to routinely synthesize peptides of twenty or more amino acids in length. When preparing long peptides (e.g., longer than forty amino acids) it may prove advantageous to generate the peptide in a series of fragment that can be ligated by using appropriate protective groups.

As described herein, certain peptide compositions can include multiple variants of a single peptide sequence (e.g., with 2 or more different amino acids at 2 or more positions in the same sequence). While each variant in a given set could be synthesized individually, it will typically be advantageous to synthesize a subset or the entire set of variants in a single synthesis. In various embodiments, this can be achieved using a split-combine method. Thus, if the set of variants has been designed to include one of two different amino acids at a given position, the resin can be split into two equal parts and each part can be coupled with one of the two amino acids to form two distinctive peptide chains. Once the coupling has been completed the two parts can be recombined so that the subsequent amino acid can be added to both chains. Alternatively, a one-pot method may be used wherein the two alternative amino acids are added to the same reaction. Typically the two amino acids will be added in equimolar amounts; however in certain circumstances (e.g., if their respective reaction kinetics differ significantly) non-equimolar amounts may be preferred. The peptide reaction can then be monitored for completeness using a Kaiser ninhydrin test.

The following references describe some exemplary methods for preparing peptide mixtures: Houghten, Proc. Natl. Acad. Sci. USA 82:5131 (1985); Geysen et al, Proc. Natl. Acad. Sci. USA 81:3998 (1984) and U.S. Pat. No. 5,010,175.

### IV. Adjuvants

In some embodiments, immunogenic compositions may include one or more adjuvants. As is well known in the art, adjuvants are agents that enhance immune responses. Adjuvants are well known in the art (e.g., see “Vaccine Design: The Subunit and Adjuvant Approach”, Pharmaceutical Biotechnology, Volume 6, Eds. Powell and Newman, Plenum Press, New York and London, 1995).

Exemplary adjuvants include complete Freund's adjuvant (CFA), incomplete Freund's adjuvant (IFA), squalene, squalane and alum (aluminum hydroxide), which are materials well known in the art, and are available commercially from several sources. In some embodiments, aluminum or calcium salts (e.g., hydroxide or phosphate salts) may be used as adjuvants. Alum (aluminum hydroxide) has been used in many existing vaccines. Typically, about 40 to about 700 μg of aluminum can be included per dose.

In various embodiments, oil-in-water emulsions or water-in-oil emulsions can also be used as adjuvants. For example, the oil phase may include squalene or squalane and a surfactant. In various embodiments, non-ionic surfactants such as the mono- and di-C₁₂-C₂₄-fatty acid esters of sorbitan and mannide may be used. The oil phase preferably comprises about 0.2 to about 15% by weight of the immunogenic peptide(s) (e.g., about 0.2 to 1%). PCT Publication No. WO 95/17210 describes exemplary emulsions.

The adjuvant designated QS21 is an immunologically active saponin fractions having adjuvant activity derived from the bark of the South American tree Quillaja Saponaria Molina, and the method of its production is disclosed in U.S. Pat. No. 5,057,540. Semi-synthetic and synthetic derivatives of Quillaja Saponaria Molina saponins are also useful, such as those described in U.S. Pat. Nos. 5,977,081 and 6,080,725.

TLRs are a family of proteins homologous to the Drosophila Toll receptor, which recognize molecular patterns associated with pathogens and thus aid the body in distinguishing between self and non-self molecules. Substances common in viral pathogens are recognized by TLRs as pathogen-associated molecular patterns. For example, TLR-3 recognizes patterns in double-stranded RNA, TLR-4 recognizes patterns in lipopolysaccharides while TLR-7/8 recognize patterns containing adenosine in viral and bacterial RNA and DNA. When a TLR is triggered by such pattern recognition, a series of signaling events occurs that leads to inflammation and activation of innate and adaptive immune responses. A number of synthetic ligands containing the molecular patterns recognized by various TLRs are being developed as adjuvants and may be included in an immunogenic composition as described herein.

For example, polyriboinosinic:polyribocytidylic acid or poly(I:C) (available from InvivoGen of San Diego, Calif.) is a synthetic analog of double-stranded RNA (a molecular pattern associated with viral infection) and an exemplary adjuvant that is an agonist for TLR-3 (e.g., see Field et al., Proc. Natl. Acad. Sci. USA 58:1004 (1967) and Levy et al., Proc. Natl. Acad. Sci. USA 62:357 (1969)). Example 11 below demonstrates the benefits of using this adjuvant with an exemplary oral peptide composition. In some embodiments, poly(I:C) may be combined with other agents to improve stability (e.g., by reducing degradation via the activity of RNAses). For example, U.S. Pat. Nos. 3,952,097, 4,024,241 and 4,349,538 describe poly(I:C) complexes with poly-L-lysine. The addition of poly-arginine to poly(I:C) has also been shown to reduce degradation via the activity of RNAses. U.S. Patent Publication No. 20090041809 describes double-stranded nucleic acids with one or more than one locked nucleic acid (LNA) nucleosides that can act as TLR-3 agonists. Those skilled in the art will be able to identify other suitable TLR-3 agonist adjuvants.

Attenuated lipid A derivatives (ALD) such as monophosphoryl lipid A (MPL) and 3-deacyl monophosphoryl lipid A (3D-MPL) are exemplary adjuvants that are agonists for TLR-4. ALDs are lipid A-like molecules that have been altered or constructed so that the molecule displays lesser or different of the adverse effects of lipid A. These adverse effects include pyrogenicity, local Shwarzman reactivity and toxicity as evaluated in the chick embryo 50% lethal dose assay (CELD₅₀). MPL and 3D-MPL are described in U.S. Pat. Nos. 4,436,727 and 4,912,094, respectively. MPL was originally derived from lipid A, a component of enterobacterial lipopolysaccharides (LPS), a potent but highly toxic immune system modulator. 3D-MPL differs from MPL in that the acyl residue that is ester linked to the reducing-end glucosamine at position 3 has been selectively removed. It will be appreciated that MPL and 3D-MPL may include a mixture of a number of fatty acid substitution patterns, i.e., heptaacyl, hexaacyl, pentaacyl, etc., with varying fatty acid chain lengths. Thus, various forms of MPL and 3D-MPL, including mixtures thereof, are encompassed by the present disclosure.

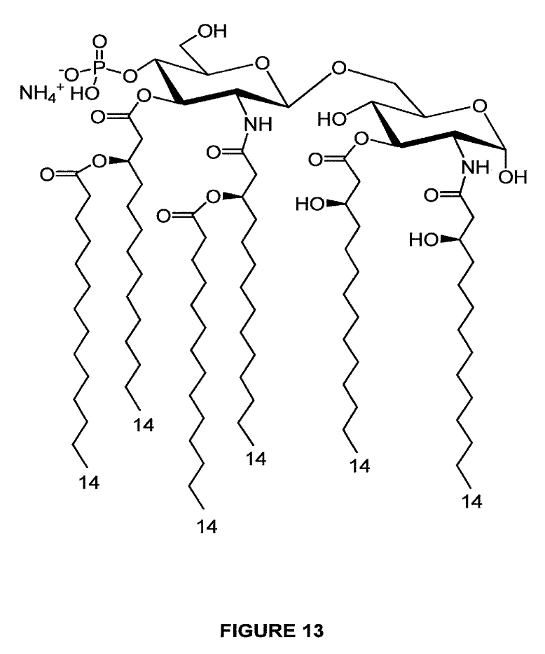

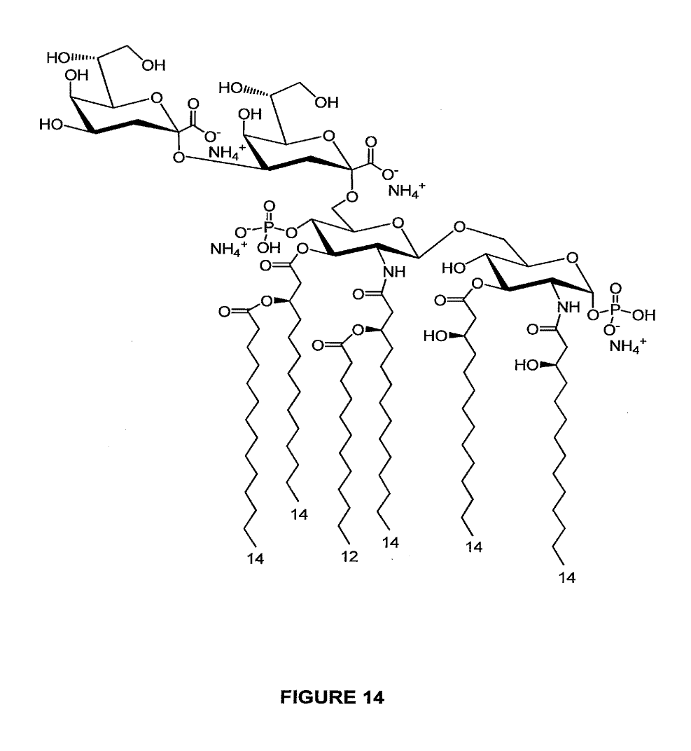

In some embodiments these ALDs may be combined with trehalosedimycolate (TDM) and cell wall skeleton (CWS), e.g., in a 2% squalene/Tween™ 80 emulsion (e.g., see GB Patent No. 2122204). MPL is available from Avanti Polar Lipids, Inc. of Alabaster, Ala. as PHAD (phosphorylated hexaacyl disaccharide). FIG. 13 shows a chemical structure of PHAD. Example 13 below demonstrates the benefits of using this adjuvant with an exemplary parenteral peptide composition. The structure of di[3-deoxy-D-manno-octulosonyl]-lipid A (ammonium salt) another exemplary TLR-4 agonist adjuvant is shown in FIG. 14 (also from Avanti Polar Lipids, Inc. of Alabaster, Ala.). Those skilled in the art will be able to identify other suitable TLR-4 agonist adjuvants. For example, other lipopolysaccharides have been described in WO 98/01139; U.S. Pat. No. 6,005,099 and EP Patent No. 729473.

Imiquimod (1-isobutyl-1H-imidazo[4,5-c]quinolin-4-amine) is a small molecule agonist of TLR-7/8 which may also be advantageously included in an immunogenic composition as described herein.

### V. Vesicles

In some embodiments, one or more peptides in a composition may be combined with a vesicle. As is well known in the art, vesicles generally have an aqueous compartment enclosed by one or more bilayers which include amphipathic molecules (e.g., fatty acids, lipids, steroids, etc.). The one or more peptides may be present in the aqueous core of the vesicle. Depending on its hydrophobicity, a peptide may also be associated with a bilayer (e.g., through hydrophobic interactions and/or hydrogen or ionic bonds). It is to be understood that any vesicle may be used with an immunogenic composition as described herein and that the amphipathic molecules of the bilayer may be ionic and/or non-ionic.

In some embodiments, the vesicle may comprise a non-ionic amphiphile (e.g., a non-ionic surfactant). Any non-ionic surfactant with appropriate amphipathic properties may be used to form such a vesicle. Without limitation, examples of suitable surfactants include ester-linked surfactants based on glycerol. Such glycerol esters may comprise one of two higher aliphatic acyl groups, e.g., containing at least ten carbon atoms in each acyl moiety. Surfactants based on such glycerol esters may comprise more than one glycerol unit, e.g., up to 5 glycerol units. Glycerol monoesters may be used, e.g., those containing a C₁₂-C₂₀alkanoyl or alkenoyl moiety, for example caproyl, lauroyl, myristoyl, palmitoyl, oleyl or stearoyl. An exemplary surfactant is 1-monopalmitoyl glycerol.

Ether-linked surfactants may also be used as the non-ionic surfactant. For example, ether-linked surfactants based on glycerol or a glycol having a lower aliphatic glycol of up to 4 carbon atoms, such as ethylene glycol, are suitable. Surfactants based on such glycols may comprise more than one glycol unit, e.g., up to 5 glycol units (e.g., diglycolcetyl ether and/or polyoxyethylene-3-lauryl ether). Glycol or glycerol monoethers may be used, including those containing a C₁₂-C₂₀alkanyl or alkenyl moiety, for example capryl, lauryl, myristyl, cetyl, oleyl or stearyl. Ethylene oxide condensation products that can be used include those disclosed in PCT Publication No. WO88/06882 (e.g., polyoxyethylene higher aliphatic ether and amine surfactants). Exemplary ether-linked surfactants include 1-monocetyl glycerol ether and diglycolcetyl ether.

In some embodiments, a vesicle comprising a non-ionic surfactant may further comprise an ionic amphiphile, e.g., to cause the vesicles to take on a negative charge. For example, this may help to stabilize the vesicle and provide effective dispersion. Without limitation, acidic materials such as higher alkanoic and alkenoic acids (e.g., palmitic acid, oleic acid) or other compounds containing acidic groups including phosphates such as dialkyl phosphates (e.g., dicetylphosphate, or phosphatidic acid or phosphatidyl serine) and sulphate monoesters such as higher alkyl sulphates (e.g., cetylsulphate), may all be used for this purpose. The ionic amphiphile, if present, will typically comprise, between 1 and 30% by weight of the non-ionic surfactant. For example, between 2 and 20% by weight or between 5 and 15% by weight.

In some embodiments, the vesicle components may be admixed with an appropriate hydrophobic material of higher molecular mass capable of forming a bi-layer (such as a steroid, e.g., a sterol such as cholesterol). In some embodiments, the presence of the steroid may assist in forming the bi-layer on which the physical properties of the vesicle depend. The steroid, if present, will typically comprise between 20 and 120% by weight of the non-ionic surfactant. For example, between 25 and 90% by weight or between 35 and 75% by weight.

In some embodiments, the vesicle may be a bilosome (see, e.g., U.S. Pat. No. 5,876,721). As used herein, “bilosomes” are vesicles that comprise non-ionic surfactants and transport enhancing molecules which facilitate the transport of lipid-like molecules across mucosal membranes. As described in U.S. Pat. No. 5,876,721, a variety of molecules may be used as transport enhancers. For example, cholesterol derivatives in which the C₂₃ carbon atom of the side chain carries a carboxylic acid, and/or derivatives thereof, may be used as transport enhancers. Such derivatives include, but are not limited to, the “bile acids” cholic acid and chenodeoxycholic acid, their conjugation products with glycine or taurine such as glycocholic and taurocholic acid, derivatives including deoxycholic and ursodeoxycholic acid, and salts of each of these acids.

Other transport enhancers include acyloxylated amino acids, such as acylcarnitines and salts thereof. For example, acylcarnitine containing C₆₋₂₀alkanoyl or alkenyl moieties, such as palmitoylcarnitine, may be used as transport enhancers. As used herein, the term acyloxylated amino acid is intended to cover primary, secondary and tertiary amino acids as well as α, β, and γ amino acids. Acylcarnitines are examples of acyloxylated γ amino acids.

It is to be understood that bilosomes which are included in an immunogenic composition may comprise more than one type of transport enhancer, e.g., one or more different bile salts and one or more acylcarnitines.

It is also to be understood that bilosomes may also incorporate an ionic amphiphile, e.g., to cause the bilosomes to take on a negative charge. For example, this may help to stabilize the bilosomes and provide effective dispersion. Without limitation, acidic materials such as higher alkanoic and alkenoic acids (e.g., palmitic acid, oleic acid) or other compounds containing acidic groups including phosphates such as dialkyl phosphates (e.g., dicetylphospate, or phosphatidic acid or phosphatidyl serine) and sulphate monoesters such as higher alkyl sulphates (e.g., cetylsulphate), may all be used for this purpose.

In some embodiments, the bilosome components may be admixed with an appropriate hydrophobic material of higher molecular mass capable of forming a bi-layer (such as a steroid, e.g., a sterol such as cholesterol). In some embodiments, the presence of the steroid may assist in forming the bi-layer on which the physical properties of the bilosome depend.

The transport enhancer(s) present within the bilosomes will generally be present in amount of between 40 and 400% percent by weight of the non-ionic surfactant (e.g., between 60 and 100% by weight or between 70 and 90% by weight). The steroid, if present, will typically comprise between 20 and 120% by weight of the non-ionic surfactant (e.g., between 25 and 90% by weight or between 35 and 75% by weight). The ionic amphiphile, if present, will typically comprise, between 1 and 30% by weight of the non-ionic surfactant (e.g., between 2 and 20% by weight or between 5 and 15% by weight).

There are many known techniques for preparing vesicles comprising non-ionic surfactants, e.g., those referred to in PCT Publication No. WO1993/019781. It will be appreciated that any of these known methods may be used to prepare suitable vesicles and that bilosomes may be prepared by modifying any of these techniques. An exemplary technique is the rotary film evaporation method, in which a film of non-ionic surfactant is prepared by rotary evaporation from an organic solvent, e.g., a hydrocarbon or chlorinated hydrocarbon solvent such as chloroform, e.g., see Russell and Alexander, J. Immunol. 140:1274 (1988). The resulting thin film is then rehydrated in bicarbonate buffer in the presence of the transport enhancer.

Another method for the production of vesicles that can be adapted for preparation of bilosomes is that disclosed by Collins et al., J. Pharm. Pharmacol. 42:53 (1990). This method involves melting a mixture of the non-ionic surfactant, steroid (if used) and ionic amphiphile (if used) and hydrating with vigorous mixing in the presence of aqueous buffer. The transport enhancer can be incorporated into the vesicles, either by being included with the other constituents in the melted mixture (i.e., by co-melting) or concomitantly during the process used to entrap the peptides.

Another method involves hydration in the presence of shearing forces. An apparatus that can be used to apply such shearing forces is a well known, suitable equipment (see, e.g., PCT Publication No. WO88/06882). Sonication and ultra-sonication are also effective means to form the vesicles or to alter their particle size.

The one or more peptides may be combined with vesicles in any manner. For example, in the rotary film evaporation technique, this can be achieved by hydration of the film in the presence of peptides together with the transport enhancer. In other methods, the one or more peptides may be combined with preformed vesicles by a dehydration-rehydration method in which peptides present in the aqueous phase are entrapped by flash freezing followed by lyophilisation, e.g., see Kirby and Gregoriadis, Biotechnology 2:979 (1984). Alternatively a freeze thaw technique may be used in which vesicles are mixed with the peptides and repeatedly flash frozen in liquid nitrogen, and warmed to a temperature of the order of, e.g., 60° C. (i.e., above the transition temperature of the relevant surfactant), e.g., see Pick, Arch. Biochem. Biophys. 212:195 (1981). In addition to entrapping peptides, the dehydration-rehydration method and freeze-thaw technique are also capable of concomitantly incorporating additional transport enhancers into the vesicles.

In each of these methods, the suspension of vesicle components may be extruded several times through microporous polycarbonate membranes at an elevated temperature sufficient to maintain the vesicle-forming mixture in a molten condition. This has the advantage that vesicles having a uniform size may be produced. Vesicles (including bilosomes) that may be used in accordance with the invention may be of any diameter. In some embodiments, the composition may include vesicles with diameters in the range of about 10 nm to about 10 μm. In some embodiments, vesicles are of diameters between about 100 nm to about 5 μm. In some embodiments, vesicles are of diameters between about 500 nm to about 2 μm. In some embodiments, vesicles are of diameters between about 800 nm to about 1.5 μm.

### VI. Dosage and Administration

The methods of this invention are useful for treating influenza in humans including adults and children. In general however they may be used with any animal. In some embodiments, the methods herein may be used for veterinary applications, e.g., canine and feline applications. If desired, the methods herein may also be used with farm animals, such as ovine, avian, bovine, porcine and equine breeds.

Compositions described herein will generally be administered in such amounts and for such a time as is necessary or sufficient to induce an immune response. Dosing regimens may consist of a single dose or a plurality of doses over a period of time. The exact amount of a peptide composition to be administered may vary from subject to subject and may depend on several factors. Thus, it will be appreciated that, in general, the precise dose used will be as determined by the prescribing physician and will depend not only on the weight of the subject and the route of administration, but also on the age of the subject and the severity of the symptoms and/or the risk of infection. In some embodiments, the dose of peptide in an immunogenic composition may range from about 0.01 to 50 mg. For example, in some embodiments the range may be between 0.1 and 5 mg, e.g., between 0.1 and 2 mg. Example 14 describes exemplary administration schemes that involve two consecutive intramuscular injections of 0.2 or 1 mg peptide doses (on Day 0 and Day 28).

In general, the compositions may be administered to a subject by any route. The results in the Examples demonstrate that the immunogenic peptide compositions described herein can induce a protective response when administered via the traditional parenteral route but also orally. Thus, in some embodiments, the compositions may be administered orally (including buccally, sublingually and by gastric lavage or other artificial feeding means). Such oral delivery may be accomplished using solid or liquid compositions, for example in the form of tablets, capsules, multi-particulates, gels, films, ovules, elixirs, solutions, suspensions, etc. In some embodiments, when using a liquid composition, the composition may be administered in conjunction with a basic composition (e.g., a bicarbonate solution) in order to neutralize the stomach pH. In some embodiments, the basic composition may be administered before and/or after the peptide composition. In some embodiments, the basic composition may be combined with the peptide composition prior to administration or taken at the same time as the peptide composition.

It will be appreciated that the oral route is particularly desirable in light of the advantages of oral delivery over any form of injection. It will also be appreciated that the results are surprising in light of the fact that all known influenza vaccines have so far been administered parenterally. The ability to induce a protective response by oral delivery is particularly unexpected in light of the fact that the immunogenic compositions described herein include peptides that are much shorter than the standard protein antigens that are used in current influenza vaccines. Indeed, there has long been a prejudice in the art against both the use of short peptides as immunogens and oral delivery of protein vaccines.

In some embodiments, oral compositions comprise one or more peptides, bilosomes and an adjuvant. In some embodiments, the adjuvant is a TLR-3 agonist. In some embodiments, the adjuvant may be mixed with the bilosomes. In some embodiments, the adjuvant may be associated with the bilosomes (e.g., by incorporating the adjuvant with the one or more peptides and/or bilosome components during the process used to make the bilosomes).

In some embodiments, the compositions may be formulated for delivery parenterally, e.g., by injection. In such embodiments, administration may be, for example, intravenous, intramuscular, intradermal, or subcutaneous, or via by infusion or needleless injection techniques. For such parenteral administration, the compositions may be prepared and maintained in conventional lyophylized compositions and reconstituted prior to administration with a pharmaceutically acceptable saline solution, such as a 0.9% saline solution. The pH of the injectable composition can be adjusted, as is known in the art, with a pharmaceutically acceptable acid, such as methanesulfonic acid. Other acceptable vehicles and solvents that may be employed include Ringer's solution and U.S.P. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable compositions can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, parenteral compositions comprise one or more peptides, vesicles that comprise non-ionic surfactants and an adjuvant. In some embodiments, the adjuvant is a TLR-4 agonist. In some embodiments, the adjuvant may be mixed with the vesicles. In some embodiments, the adjuvant may be associated with the vesicles (e.g., by incorporating the adjuvant with the one or more peptides and/or vesicle components during the process used to make the vesicles).

The compositions can also be administered intranasally or by inhalation and are conveniently delivered in the form of a dry powder inhaler or an aerosol spray presentation from a pressurized container, pump, spray, atomiser or nebuliser, with or without the use of a suitable propellant, e.g., dichlorodifluoromethane, trichlorofluoromethane, dichlorotetrafluoroethane, a hydrofluoroalkane, carbon dioxide or other suitable gas. In the case of a pressurized aerosol, the dosage unit may be determined by providing a valve to deliver a metered amount. The pressurized container, pump, spray, atomiser or nebuliser may contain a solution or suspension of the antibody, e.g., using a mixture of ethanol and the propellant as the solvent, which may additionally contain a lubricant, e.g., sorbitantrioleate. Capsules and cartridges (made, for example, from gelatin) for use in an inhaler or insufflator may be formulated to contain a powder mix of the peptide composition and a suitable powder base such as lactose or starch.

Compositions for rectal administration are preferably suppositories which can be prepared by mixing the peptide(s) with suitable non-irritating excipients or carriers such as cocoa butter, polyethylene glycol or a suppository wax which are solid at ambient temperature but liquid at body temperature and therefore melt in the rectal vault and release the antibodies. Retention enemas and rectal catheters can also be used as is known in the art. Viscosity-enhancing carriers such as hydroxypropyl cellulose are also certain carriers of the invention for rectal administration since they facilitate retention of the composition within the rectum. Generally, the volume of carrier that is added to the composition is selected in order to maximize retention of the composition. In particular, the volume should not be so large as to jeopardize retention of the administered composition in the rectal vault.

### VII. Exemplary Compositions

In some embodiments, the present disclosure provides immunogenic compositions that include a TLR-3 agonist adjuvant and a vesicle which comprises a non-ionic surfactant and a transport enhancer which facilitates the transport of lipid-like molecules across mucosal membranes. In some embodiments, these compositions may be administered orally. In some embodiments the TLR-3 agonist adjuvant comprises poly(I:C). In some embodiments, the transport enhancer is a bile acid, a derivative thereof or a salt of any of these (e.g., sodium deoxycholate). In some embodiments, the non-ionic surfactant is a glycerol ester (e.g., 1-monopalmitoyl glycerol). In some embodiments, the vesicle further comprises an ionic amphiphile (e.g., dicetylphospate). In some embodiments, the vesicle further comprises a steroid (e.g., cholesterol). In some embodiments, the vesicles comprise 1-monopalmitoyl glycerol, dicetylphospate, cholesterol and sodium deoxycholate. In some embodiments, the composition further comprises alum.

In some embodiments, the present disclosure provides immunogenic compositions that include a TLR-4 agonist adjuvant and a vesicle which comprises a non-ionic surfactant. In some embodiments, these compositions may be administered parenterally (e.g., by intramuscular injection). In some embodiments the TLR-4 agonist adjuvant comprises monophosphoryl lipid A or 3-deacyl monophosphoryl lipid A. In some embodiments, the non-ionic surfactant is a glycerol ester (e.g., 1-monopalmitoyl glycerol). In some embodiments, the vesicle further comprises an ionic amphiphile (e.g., dicetylphospate). In some embodiments, the vesicle further comprises a steroid (e.g., cholesterol). In some embodiments, the vesicles comprise 1-monopalmitoyl glycerol, dicetylphospate and cholesterol. In some embodiments, the composition further comprises alum.

In some embodiments, the aforementioned immunogenic compositions include a first peptide comprising a region having at least 80% homology with 20-100 contiguous amino acids of SEQ ID NO. 16, wherein the first peptide includes fewer than 100 contiguous amino acids from a type A influenza hemagglutinin protein; a second peptide comprising an amino acid sequence of SEQ ID NO. 17; a third peptide comprising a region having at least 80% homology with at least 20 contiguous amino acids from positions 2-49 or 68-121 of SEQ ID NO. 18, wherein the third peptide includes fewer than 100 contiguous amino acids from a type B influenza hemagglutinin protein; and a fourth peptide comprising an amino acid sequence of SEQ ID NO. 14, wherein the fourth peptide includes fewer than 20 contiguous amino acids from a type A influenza nucleoprotein.

In some embodiments, the first peptide comprises at least 40 contiguous amino acids of SEQ ID NO. 6. In some embodiments, the first peptide comprises amino acids 1-88 of SEQ ID NO. 6.

In some embodiments, the second peptide comprises an amino acid sequence of SEQ ID NO. 11. In some embodiments the composition comprises 2ⁿ different second peptides each comprising a different amino acid sequence of SEQ ID NO. 11, where n=1-4. In some embodiments n=4.

In some embodiments, the third peptide comprises a region having at least 90% homology with at least 20 contiguous amino acids from positions 2-49 or 68-121 of SEQ ID NO. 18. In some embodiments, the third peptide comprises a first region having at least 80% homology with at least 20 contiguous amino acids from positions 2-49 and a second region having at least 80% homology with at least 20 contiguous amino acids from positions 68-121 of SEQ ID NO. 18. In some embodiments the composition comprises two different third peptides, one of which comprises a region having at least 80% homology with at least 20 contiguous amino acids from positions 2-49 of SEQ ID NO. 18 while the other comprises a region having at least 80% homology with at least 20 contiguous amino acids from positions 68-121 of SEQ ID NO. 18. In some embodiments, the composition comprises two different third peptides, one of which comprises an amino acid sequence of SEQ ID NO. 12 while the other comprises an amino acid sequence of SEQ ID NO. 13.

In some embodiments, the composition comprises 2ⁿ different fourth peptides each comprising a different amino acid sequence of SEQ ID NO. 14, where n=1-4. In some embodiments n=4.

In some embodiments, the first peptide comprises amino acids 1-88 of SEQ ID NO. 6; the composition comprises 2ⁿ different second peptides each comprising a different amino acid sequence of SEQ TD NO. 11, where n=4; the composition comprises two different third peptides, one of which comprises an amino acid sequence of SEQ ID NO. 12 while the other comprises an amino acid sequence of SEQ ID NO. 13; and the composition comprises 2^{m} different fourth peptides each comprising a different amino acid sequence of SEQ ID NO. 14, where m=4.

In some embodiments, the aforementioned compositions are used to treat an individual suffering from, or at risk for, seasonal influenza.

In some embodiments, the aforementioned compositions are used to treat an individual suffering from, or at risk for, influenza caused by an influenza A (H1N1) virus of swine origin.

### EXAMPLES

The following examples describe some exemplary modes of making and practicing certain compositions and methods that are described herein. It should be understood that these examples are for illustrative purposes only and are not meant to limit the scope of the compositions and methods described herein.

### Example 1

### Peptide Synthesis

All peptides described herein were synthesized by solid phase peptide synthesis (SPPS). Generally, the C-terminal amino acid was attached to a cross-linked polystyrene (or PEG-based) resin via an acid labile bond with a linker molecule. This resin is insoluble in the solvents used for synthesis, making it relatively simple and fast to wash away excess reagents and by-products. The N-terminus was protected with an Fmoc group, which is stable in acid, but removable by base. Any side chain functional groups were protected with base stable, acid labile groups. The synthesis was then based on the incorporation of N-α-Fmoc protected amino acids into the growing peptide chain with one end of the chain remaining attached to the solid phase.

The following is an exemplary SPPS method that was used to make the peptides described herein. To begin each coupling, the Fmoc group on the NovaPEG resin bound amino acid/peptide was removed with 20% piperidine in N,N-dimethylformamide (DMF). It was then rinsed and a protected amino acid that had been activated at its α-carboxyl group by 2-(1H-Benzotriazole-1-yl)-1,1,3,3-tetramethyluronium tetrafluoroborate was added. The activated amino acid and the resin-bound amino acid were allowed to react in the presence of base to form a new peptide bond. While peptides were synthesized by stepwise method, all soluble reagents, such as the coupling reagent, 2-(1H-Benzotriazole-1-yl)-1,1,3,3-tetramethyluronium tetrafluoroborate (TBTU), base, diisopropylethyl amine (DIPEA) and de-blocking reagent, piperidine, were removed from the peptide-solid support matrix by filtration and rinsed at the end of each coupling step. This process was repeated until the desired peptide was assembled on the resin. After cleavage from the resin and deprotection of protective groups from the peptides by a cleavage solution containing at least 85% of Trifluoro acetic acid (TFA) a crude peptide was collected by precipitation from ether, followed by centrifugation. The peptides were then purified and characterized by HPLC and mass spectroscopy.

As described herein, certain peptide compositions can include multiple variants of a single peptide sequence (e.g., with 2 or more different amino acids at 2 or more positions in the same sequence). While each variant in a given set could be synthesized individually, it will typically be advantageous to synthesize a subset or the entire set of variants in a single synthesis. For example, this can be achieved using a split-combine method. Thus, if the set of variants has been designed to include one of two different amino acids at a given position, the resin can be split into two equal parts and each part can be coupled with one of the two amino acid to form two distinctive peptide chains. Once the coupling has been completed the two parts can be recombined so that the subsequent amino acid can be added to both chains. Alternatively, a one-pot method may be used wherein the two alternative amino acids are added to the same reaction. Typically the two amino acids will be added in equimolar amounts; however in certain circumstances (e.g., if their respective reaction kinetics differ significantly) non-equimolar amounts may be preferred. The peptide reaction can then be monitored for completeness using a Kaiser ninhydrin test.

### Example 2

### Competition Micro-Neutralization Assay

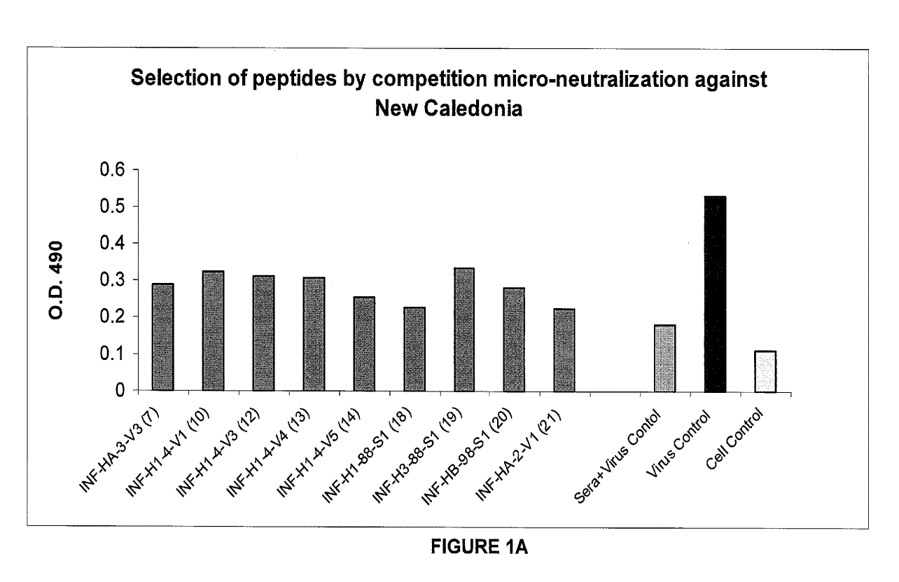

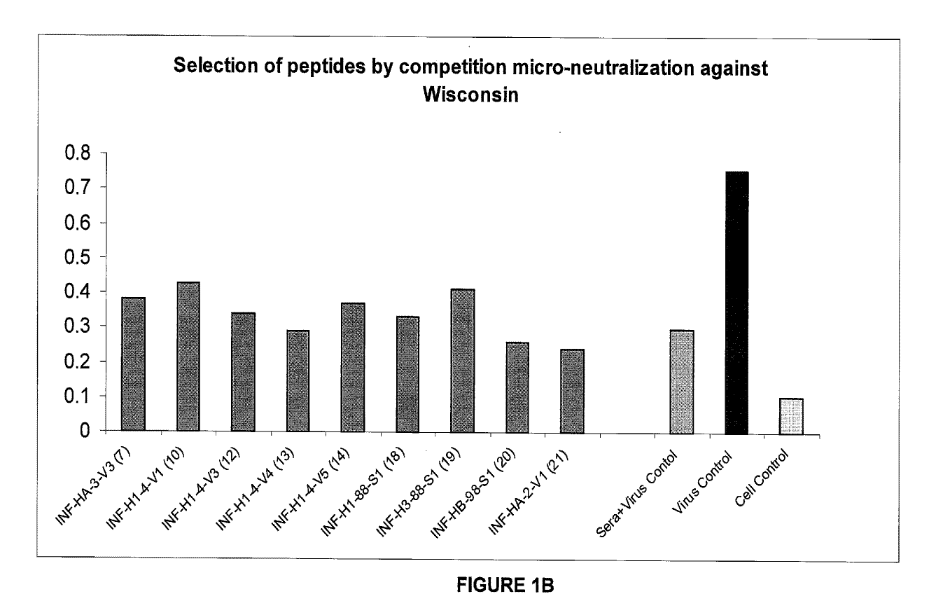

In the present Example, the ability of various peptide compositions to bind influenza neutralizing antibodies was assessed. Different peptide compositions (see FIG. 1 and Tables 1-9 below) were diluted at 80 μg/ml in Iscove's Modified Dulbecco's Medium (IMDM) and 50 μA was added to different wells in 96-well flat bottom microtitre plates (Nunc). Each peptide composition included a full complement of variants where applicable (e.g., the eight INF-HA-3-V3 peptide variants shown in Table 1, the four INF-H1-4-V4 peptide variants in Table 4, etc.). Commercial anti-influenza sera and human sera were diluted at 1/40 in IMDM and 50 μl was added to each well before the plates were incubated for 1 hr at 37° C. 500 of influenza virus strains Wisconsin (A/W is/67/05) or New Calcdonia (A/NC/20/99) containing 1×10⁵ pfu, in IMDM was added to each well and the plates were incubated for an additional 1 hr at 37° C. Subsequently, 5×10⁴ Madin Darby Canine Kidney (MDCK) cells supplemented with 2% fetal calf serum (FCS) were added to each well. Every plate contained four cell and four virus control wells. The plates were incubated at 37° C. for 18-22 hrs. Cells were fixed for 10 minutes with 50 μl of ethanol 70% per well and air-dried at room temperature. The plates were washed 5 times with PBS containing 0.05% Tween-20 and the wells were blocked with 10% FCS. After 90 minutes at 4° C., 50 μl of monoclonal antibody directed against the influenza type A nucleocapsid antigen (Chemicon) diluted 1/500 in blocking buffer was added to each well. After further incubation for 1 hr at 4° C., the plates were washed 5 times and 100 μl of goat anti mouse IgG peroxidase-conjugated affinity purified antibody ( 1/10,000) was added to each well for 30 minutes at 4° C. The plates were washed 5 times and developed by 100 μl orthophenylenediamine (OPD: Sigma, St. Louise, Mo.) and the enzyme reaction was stopped after 20 min with 1N HCl. The reaction was quantified by measuring the OD at wavelength 490 nm by ELISA plate reader (Bio-Rad). The results for compositions comprising the peptide(s) of Tables 1, 2, 3, 4, 5, 6, 7, 8 and 9 are shown in FIG. 1.

As shown in FIG. 1, addition of commercial or immune sera can inhibit influenza virus infection of cells in vitro, which is quantified by ELISA by optical density (OD) measurements. Thus, an OD value of approximately 0.6 (see Virus control) is reduced to approximately 0.2 in the presence of immune sera (see Virus+Sera control). Incubation of various peptide compositions with the sera allows the peptides to bind to some of the neutralizing antibodies present in the sera and thereby reverse their neutralizing activity (e.g., OD values of approximately 0.3 or higher).

TABLE 1
SEQ ID NO. 1

| Peptide Name | Sequence |
|---|---|
| Consensus | GSRPXVREDGGLPQSXRISIXWTIVKPG |
| Substitution # 1 | ----D----------G----D------- |
| Substitution # 2 | ----W----------S----Y------- |
| INF-HA-3-V3/1 | GSRPDVREDGGLPQSGRISIDWTIVKPG |
| INF-HA-3-V3/2 | GSRPDVREDGGLPQSGRISIYWTIVKPG |
| INF-HA-3-V3/3 | GSRPDVREDGGLPQSSRISIDWTIVKPG |
| INF-HA-3-V3/4 | GSRPDVREDGGLPQSSRISIYWTIVKPG |
| INF-HA-3-V3/5 | GSRPWVREDGGLPQSGRISIDWTIVKPG |
| INF-HA-3-V3/6 | GSRPWVREDGGLPQSGRISIYWTIVKPG |
| INF-HA-3-V3/7 | GSRPWVREDGGLPQSSRISIDWTIVKPG |
| INF-HA-3-V3/8 | GSRPWVREDGGLPQSSRISIYWTIVKPG |

TABLE 2
SEQ ID NO. 2

| Peptide Name | Sequence |
|---|---|
| Consensus | XTGVSASCSHNGXSSFYXNLLWLTGK |
| Substitution # 1 | V-----------K----R-------- |
| Substitution # 2 | T-----------E----K-------- |
| INF-H1-4-V1/1 | VTGVSASCSHNGKSSFYRNLLWLTGK |
| INF-H1-4-V1/2 | VTGVSASCSHNGKSSFYKNLLWLTGK |
| INF-H1-4-V1/3 | VTGVSASCSHNGESSFYRNLLWLTGK |
| INF-H1-4-V1/4 | VTGVSASCSHNGESSFYKNLLWLTGK |
| INF-H1-4-V1/5 | TTGVSASCSHNGKSSFYRNLLWLTGK |
| INF-H1-4-V1/6 | TTGVSASCSHNGKSSFYKNLLWLTGK |
| INF-H1-4-V1/7 | TTGVSASCSHNGESSFYRNLLWLTGK |
| INF-H1-4-V1/8 | TTGVSASCSHNGESSFYKNLLWLTGK |

TABLE 3
SEQ ID NO. 3

| Peptide Name | Sequence |
|---|---|
| Consensus | HYSRXFTPEIXKRPKVRXQEGRINYYWILLEPG |
| Substitution # 1 | ----R-----A------D--------------- |
| Substitution # 2 | ----K-----T------N--------------- |
| INF-H1-4-V3/1 | HYSRRFTPEIAKRPKVRDQEGRINYYWTLLEPG |
| INF-H1-4-V3/2 | HYSRRFTPEIAKRPKVRNQEGRINYYWTLLEPG |
| INF-H1-4-V3/3 | HYSRRFTPEITKRPKVRDQEGRINYYWTLLEPG |
| INF-H1-4-V3/4 | HYSRRFTPEITKRPKVRNQEGRINYYWTLLEPG |
| INF-H1-4-V3/5 | HYSRKFTPEIAKRPKVRDQEGRINYYWTLLEPG |
| INF-H1-4-V3/6 | HYSRKFTPEIAKRPKVRNQEGRINYYWTLLEPG |
| INF-H1-4-V3/7 | HYSRKFTPEITKRPKVRDQEGRINYYWTLLEPG |
| INF-H1-4-V3/8 | HYSRKFTPEITKRPKVRNQEGRINYYWTLLEPG |

TABLE 4
SEQ ID NO. 4

| Peptide Name | Sequence |
|---|---|
| Consensus | PVTIGECPKYVRSXKLRMXTGLRNIPSIQS |
| Substitution # 1 | -------------A----V----------- |
| Substitution # 2 | -------------T----A----------- |
| INF-H1-4-V4/1 | PVTIGECPKYVRSAKLRMVTGLRNIPSIQS |
| INF-H1-4-V4/2 | PVTIGECPKYVRSAKLRMATGLRNIPSIQS |
| INF-H1-4-V4/3 | PVTIGECPKYVRSTKLRMVTGLRNIPSIQS |
| INF-H1-4-V4/4 | PVTIGECPKYVRSTKLRMATGLRNIPSIQS |

TABLE 5
SEQ ID NO. 5

| Peptide Name | Sequence |
|---|---|
| Consensus | CELLISXESWSYIVEXPNPENGTCYPGXF |
| Substitution # 1 | ------K--------T-----------Y- |
| Substitution # 2 | ------R--------K-----------H- |
| INF-H1-4-V5/1 | CELLISKESWSYIVETPNPENGTCYPGYF |
| INF-H1-4-V5/2 | CELLISKESWSYIVETPNPENGTCYPGHF |
| INF-H1-4-V5/3 | CELLISKESWSYIVEKPNPENGTCYPGYF |
| INF-H1-4-V5/4 | CELLISKESWSYIVEKPNPENGTCYPGHF |
| INF-H1-4-V5/5 | CELLISRESWSYIVETPNPENGTCYPGYF |
| INF-H1-4-V5/6 | CELLISRESWSYIVETPNPENGTCYPGHF |
| INF-H1-4-V5/7 | CELLISRESWSYIVEKPNPENGTCYPGYF |
| INF-H1-4-V5/8 | CELLISRESWSYIVEKPNPENGTCYPGHF |

TABLE 6
SEQ ID NO. 6

| Peptide Name | Sequence |
|---|---|
| INF-H1-88-S1 | SWPNHTTTGVSASSSHNGESSFYKNLLWLTGKNGL |
| | YPNLSKSYANNKEKEVLVLWGVHHPPNIGDQRALY |
| | HKENAYVSVVSIIFEANG |

TABLE 7
SEQ ID NO. 7

| Peptide Name | Sequence |
|---|---|
| INF-H3-88-S1 | NWTGVTQNGTSSSSKRRSNNSFFSRLNWLTHLKFK |
| | YPALNTMPNNEKFDKLYIWGVHHPVTDNDQIFLYA |
| | QASGRITVSTLLINSTG |

TABLE 8
SEQ ID NO. 8

| Peptide Name | Sequence |
|---|---|
| INF-HB-98-S1 | NAEKAPGGPYKIGTSGSSPNVTNGNGFFATMAWAV |
| | PKNDNNKTATNSLTIEVPYISTEGEDQITIWGFHS |
| | DNETQMAKLYGDSKPQKFTSSAGTITYQ |

TABLE 9
SEQ ID NO. 9

| Peptide Name | Sequence |
|---|---|
| Consensus | YACKXGGKSSGSSYPVLXXXY |
| Substitution # 1 | ----R------------KVS- |
| Substitution # 2 | ----Y------------SRT- |
| INF-HA-2-V1/1 | YACKRGGKSSGSSYPVLKVSY |
| INF-HA-2-V1/2 | YACKRGGKSSGSSYPVLKVTY |
| INF-HA-2-V1/3 | YACKRGGKSSGSSYPVLKRSY |
| INF-HA-2-V1/4 | YACKRGGKSSGSSYPVLKRTY |
| INF-HA-2-V1/5 | YACKRGGKSSGSSYPVLSVSY |
| INF-HA-2-V1/6 | YACKRGGKSSGSSYPVLSVTY |
| INF-HA-2-V1/7 | YACKRGGKSSGSSYPVLSRSY |
| INF-HA-2-V1/8 | YACKRGGKSSGSSYPVLSRTY |
| INF-HA-2-V1/9 | YACKYGGKSSGSSYPVLKVSY |
| INF-HA-2-V1/10 | YACKYGGKSSGSSYPVLKVTY |
| INF-HA-2-V1/11 | YACKYGGKSSGSSYPVLKRSY |
| INF-HA-2-V1/12 | YACKYGGKSSGSSYPVLKRTY |
| INF-HA-2-V1/13 | YACKYGGKSSGSSYPVLSVSY |
| INF-HA-2-V1/14 | YACKYGGKSSGSSYPVLSVTY |
| INF-HA-2-V1/15 | YACKYGGKSSGSSYPVLSRSY |
| INF-HA-2-V1/16 | YACKYGGKSSGSSYPVLSRTY |

### Example 3

### ELISPOT Assay

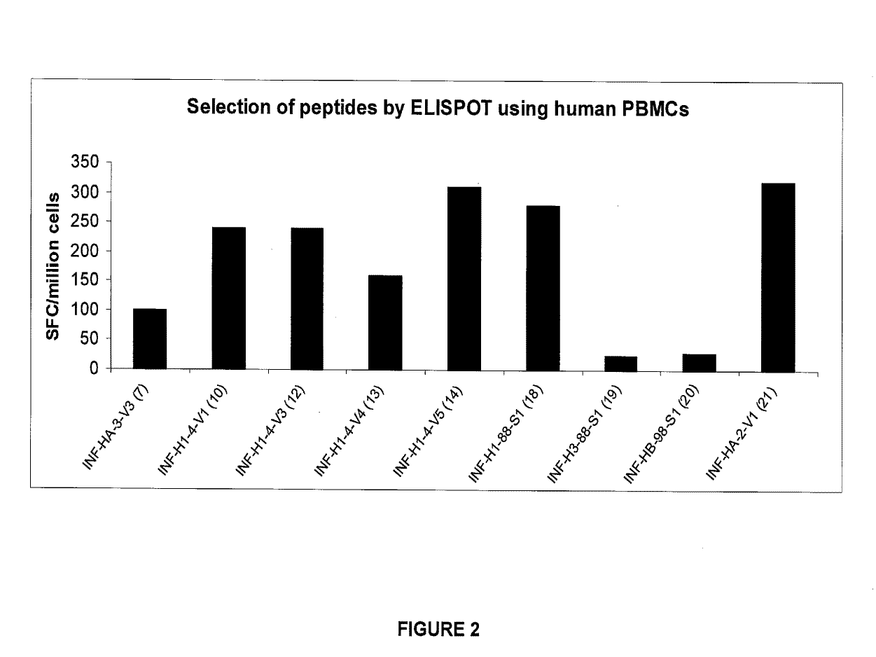

In this Example, the immunogenicity of the peptide compositions of Example 1 was tested by measuring activation (as measured by IFNγ output) of human PBMCs in an ELISPOT assay. Multiscreen-HTS plates (Millipore, Bedford, Mass.) were coated with 10 μg/ml of anti-mouse IFNγ antibody (mAb AN18, Mabtech, Mariemont, Ohio) in PBS overnight at 4° C. The plates were then washed with PBS and blocked with IMDM containing 10% FCS and 100 U/ml penicillin/streptomycin for 1 hr at room temperature. The medium was removed and 2×10⁵ peripheral blood mononuclear cell (PBMCs) from influenza-vaccinated human subjects (200 μl/well) mixed with peptide compositions (40 μg/ml, see FIG. 2 and Tables 1-9) were added to each well for 2 days. After incubation, cells were removed, washed with PBS+0.05% Tween 20 and incubated with 1 μg/ml of biotinylated anti-mouse IFNγ antibody (mAb R4-6A2-Biotin, Mabtech) for 2 hrs at room temperature. After washing, 100 μl/well of 1/1000 Streptavidin-ALP-PQ (Mabtech) in PBS+0.5% FCS was added and incubated for 1 hr at room temperature. The plates were washed as above and developed with 100 μl per well BCIP/NBT alkaline phosphatase (Moss Inc) for 20 minutes at room temperature. The reaction was stopped by rinsing the plates with tap water. The numbers of spots in each well were magnified and counted. The results for compositions comprising the peptide(s) of Tables 1, 2, 3, 4, 5, 6, 7, 8 and 9 are shown in FIG. 2.

As shown in FIG. 2, certain peptide compositions elicited a strong response in this assay, confirming that these compositions were immunogenic.

### Example 4

### Other Peptides

Tables 10-14 in this Example describe other exemplary peptides that may be used in the compositions and methods described herein.

TABLE 10
SEQ ID NO. 10

| Peptide Name | Sequence |
|---|---|
| Consensus | YACKXGGKSSGSSYPVLXVXX |
| Substitution # 1 | ----R------------N-SY |
| Substitution # 2 | ----H------------S-TM |
| INF-HA-1-V1/1 | YACKRGGKSSGSSYPVLNVSY |
| INF-HA-1-V1/2 | YACKRGGKSSGSSYPVLNVSM |
| INF-HA-1-V1/3 | YACKRGGKSSGSSYPVLNVTY |
| INF-HA-1-V1/4 | YACKRGGKSSGSSYPVLNVTM |
| INF-HA-1-V1/5 | YACKRGGKSSGSSYPVLSVSY |
| INF-HA-1-V1/6 | YACKRGGKSSGSSYPVLSVSM |
| INF-HA-1-V1/7 | YACKRGGKSSGSSYPVLSVTY |
| INF-HA-1-V1/8 | YACKRGGKSSGSSYPVLSVTM |
| INF-HA-1-V1/9 | YACKHGGKSSGSSYPVLNVSY |
| INF-HA-1-V1/10 | YACKHGGKSSGSSYPVLNVSM |
| INF-HA-1-V1/11 | YACKHGGKSSGSSYPVLNVTY |
| INF-HA-1-V1/12 | YACKHGGKSSGSSYPVLNVTM |
| INF-HA-1-V1/13 | YACKHGGKSSGSSYPVLSVSY |
| INF-HA-1-V1/14 | YACKHGGKSSGSSYPVLSVSM |
| INF-HA-1-V1/15 | YACKHGGKSSGSSYPVLSVTY |
| INF-HA-1-V1/16 | YACKHGGKSSGSSYPVLSVTM |

TABLE 11
SEQ ID NO. 11

| Peptide Name | Sequence |
|---|---|
| Consensus | YASKXGGKSSGSSYPVLXVXX |
| Substitution # 1 | ----R------------N-SY |
| Substitution # 2 | ----H------------S-TM |
| INF-HA-1-V1(C3S)/1 | YASKRGGKSSGSSYPVLNVSY |
| INF-HA-1-V1(C3S)/2 | YASKRGGKSSGSSYPVLNVSM |
| INF-HA-1-V1(C3S)/3 | YASKRGGKSSGSSYPVLNVTY |
| INF-HA-1-V1(C3S)/4 | YASKRGGKSSGSSYPVLNVTM |
| INF-HA-1-V1(C3S)/5 | YASKRGGKSSGSSYPVLSVSY |
| INF-HA-1-V1(C3S)/6 | YASKRGGKSSGSSYPVLSVSM |
| INF-HA-1-V1(C3S)/7 | YASKRGGKSSGSSYPVLSVTY |
| INF-HA-1-V1(C3S)/8 | YASKRGGKSSGSSYPVLSVTM |
| INF-HA-1-V1(C3S)/9 | YASKHGGKSSGSSYPVLNVSY |
| INF-HA-1-V1(C3S)/10 | YASKHGGKSSGSSYPVLNVSM |
| INF-HA-1-V1(C3S)/11 | YASKHGGKSSGSSYPVLNVTY |
| INF-HA-1-V1(C3S)/12 | YASKHGGKSSGSSYPVLNVTM |
| INF-HA-1-V1(C3S)/13 | YASKHGGKSSGSSYPVLSVSY |
| INF-HA-1-V1(C3S)/14 | YASKHGGKSSGSSYPVLSVSM |
| INF-HA-1-V1(C3S)/15 | YASKHGGKSSGSSYPVLSVTY |
| INF-HA-1-V1(C3S)/16 | YASKHGGKSSGSSYPVLSVTM |

TABLE 12

| | | SEQ |
|---|---|---|
| | | ID |
| Peptide Name | Sequence | NO. |
| 02-HB-01-S1-01 | AEKAPGGPYKIGTSGSSPNVTNGNGFFATM | 12 |
| | AWAVPKNDNNKTATNSLT | |
| 02-HB-01-S2-01 | FHSDNETQMAKLYGDSKPQKFTSSANGVTT | 13 |
| | HYVSQIGGFPNQTEDGGLPQSGRI | |

TABLE 13
SEQ ID NO. 14

| Peptide Name | Sequence |
|---|---|
| Consensus | SVQRNLPFXXXTXMA |
| Substitution # 1 | --------EKS-V-- |
| Substitution # 2 | --------DRT-I-- |
| INF-NP-1-V1/1 | SVQRNLPFEKSTVMA |
| INF-NP-1-V1/2 | SVQRNLPFEKSTIMA |
| INF-NP-1-V1/3 | SVQRNLPFEKTTVMA |
| INF-NP-1-V1/4 | SVQRNLPFEKTTIMA |
| INF-NP-1-V1/5 | SVQRNLPFERSTVMA |
| INF-NP-1-V1/6 | SVQRNLPFERSTIMA |
| INF-NP-1-V1/7 | SVQRNLPFERTTVMA |
| INF-NP-1-V1/8 | SVQRNLPFERTTIMA |
| INF-NP-1-V1/9 | SVQRNLPFDKSTVMA |
| INF-NP-1-V1/10 | SVQRNLPFDKSTIMA |
| INF-NP-1-V1/11 | SVQRNLPFDKTTVMA |
| INF-NP-1-V1/12 | SVQRNLPFDKTTIMA |
| INF-NP-1-V1/13 | SVQRNLPFDRSTVMA |
| INF-NP-1-V1/14 | SVQRNLPFDRSTIMA |
| INF-NP-1-V1/15 | SVQRNLPFDRTTVMA |
| INF-NP-1-V1/16 | SVQRNLPFDRTTIMA |

TABLE 14
SEQ ID NO. 15

| Peptide Name | Sequence |
|---|---|
| Consensus | XXXSSTLELRSXYWAI |
| Substitution # 1 | NMG--------G---- |
| Substitution # 2 | AIE--------R---- |
| INF-NP-2-V2/1 | NMGSSTLELRSGYWAI |
| INF-NP-2-V2/2 | NMGSSTLELRSRYWAI |
| INF-NP-2-V2/3 | NMESSTLELRSGYWAI |
| INF-NP-2-V2/4 | NMESSTLELRSRYWAI |
| INF-NP-2-V2/5 | NIGSSTLELRSGYWAI |
| INF-NP-2-V2/6 | NIGSSTLELRSRYWAI |
| INF-NP-2-V2/7 | NIESSTLELRSGYWAI |
| INF-NP-2-V2/8 | NIESSTLELRSRYWAI |
| INF-NP-2-V2/9 | AMGSSTLELRSGYWAI |
| INF-NP-2-V2/10 | AMGSSTLELRSRYWAI |
| INF-NP-2-V2/11 | AMESSTLELRSGYWAI |
| INF-NP-2-V2/12 | AMESSTLELRSRYWAI |
| INF-NP-2-V2/13 | AIGSSTLELRSGYWAI |
| INF-NP-2-V2/14 | AIGSSTLELRSRYWAI |
| INF-NP-2-V2/15 | AIESSTLELRSGYWAI |
| INF-NP-2-V2/16 | AIESSTLELRSRYWAI |

### Example 5

### Peptide Composition INF-61P

Peptide composition INF-61P includes a mixture of peptides as set forth in Table 15.

TABLE 15

| Peptide(s) | SEQ ID NO. |
|---|---|
| All sixteen (16) variants from Table 11 | 11¹ |
| Peptide from Table 6 | 6 |
| Peptide from Table 8 | 8 |

¹SEQ ID NO. 11 is a consensus sequence and covers 16 different variants.

### Example 6

### Peptide Composition INF-63P

Peptide composition INF-63P includes a mixture of peptides as set forth in Table 16.

TABLE 16

| Peptide(s) | SEQ ID NO. |
|---|---|
| All sixteen (16) variants from Table 11 | 11¹ |
| Peptide from Table 6 | 6 |
| Both peptides from Table 12 | 12, 13 |

¹SEQ ID NO. 11 is a consensus sequence and covers 16 different variants.

### Example 7

### Peptide Composition SFV2

Peptide composition SFV2 includes a mixture of peptides as set forth in Table 17.

TABLE 17

| Peptide(s) | SEQ ID NO. |
|---|---|
| All sixteen (16) variants from Table 11 | 11¹ |
| Peptide from Table 6 | 6 |
| Both peptides from Table 12 | 12, 13 |
| All sixteen (16) variants from Table 13 | 14² |

¹SEQ ID NO. 11 is a consensus sequence and covers 16 different variants.
²SEQ ID NO. 14 is a consensus sequence and covers 16 different variants. In certain embodiments, the peptides in SFV2 may differ from these sequences by the addition of a KSS linker at the N-terminus of each peptide which could be optionally used for lipidation at the N-terminal lysine moiety.

### Example 8

### Bilosome INF-61P Composition

This Example describes the preparation of the bilosome INF-61P composition that was used in Examples 9 and 11 for oral delivery of peptide compositions. Lipids (monopalmitoyl-glycerol, cholesterol and dicetylphosphate) were dissolved in chloroform and dried to a film, then rehydrated with the INF-61P peptides of Example 5 and sodium deoxycholate (a “bile salt”). The resulting bilosomes were evaluated for size using dynamic light scattering and for entrapment efficiency using a Ninhydrin assay. Loaded bilosomes were then adjusted in volume to produce a composition with the desired quantity of peptides. As described below, these loaded bilosomes were capable of delivering immunogenic peptide compositions to ferrets and mice even when administered orally.

### Example 9

### Oral Influenza Immunization of Ferrets with Bilosome INF-61P Composition

This Example describes in vivo testing of certain immunogenic peptide compositions in ferrets. The ferret model of influenza virus infection is the gold standard mammalian model for evaluating candidate influenza vaccines. Indeed, ferrets can be infected with clinical isolates of all major subtypes of influenza A and B and exhibit a clinical course of disease comparable to that seen in humans (including an increase in body temperature and in some cases weight loss).

A number of researchers have demonstrated that currently licensed influenza vaccines given by intramuscular (IM) injection induce neutralizing IgG antibodies that also possess hemagglutination inhibition activity. Induction of cellular immunity with these existing vaccines, and its role in protection, is unclear. As discussed below, we have shown that orally administered immunogenic peptide compositions induce IgA antibodies systemically (serum) and mucosally (rectal and nasal wash samples). Since influenza infection occurs via mucosal surfaces, an IgA response (the hallmark of a mucosal immune response) may be more efficacious than a systemic IgG response. As shown in Example 13, systemic IgG responses were obtained when the immunogenic peptide compositions were administered by standard parenteral routes (e.g., by IM injection).

For the vaccination and challenge experiments, male ferrets were selected that had been pre-screened as serum negative by the hemagglutination inhibition assay. Compositions for oral delivery were formulated so that the peptides were contained in a volume of 0.5 ml. All ferrets were bled 3 days before immunization and serum, plasma and PBMCs were collected. Ferrets receiving INF-61P compositions were immunized perorally on Days 0, 3, 14, and 17 by gastric lavage after fasting. For commercial vaccine controls, FLUVIRAL vaccine was injected IM into the quadriceps muscle on Days 0 and 17.

On Day 27, ferrets were bled for scrum and saliva and rectal washes were collected. All ferrets were then challenged intranasally with influenza virus diluted to 2×10⁶ pfu/ml with PBS (0.5 ml/nostril). For 10 days following virus challenge, nasal washes were collected and all animals were weighed and monitored daily for clinical signs of infection (body temperature measured electronically by implants within each animal). At sacrifice, animals were bled for serum, plasma, PBMCs and (in some instances) complete blood count (CBC), and spleens were collected and processed for mononuclear cells.

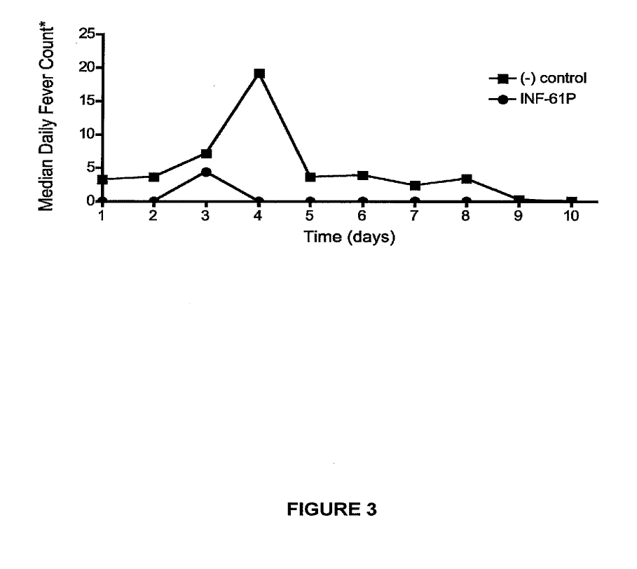

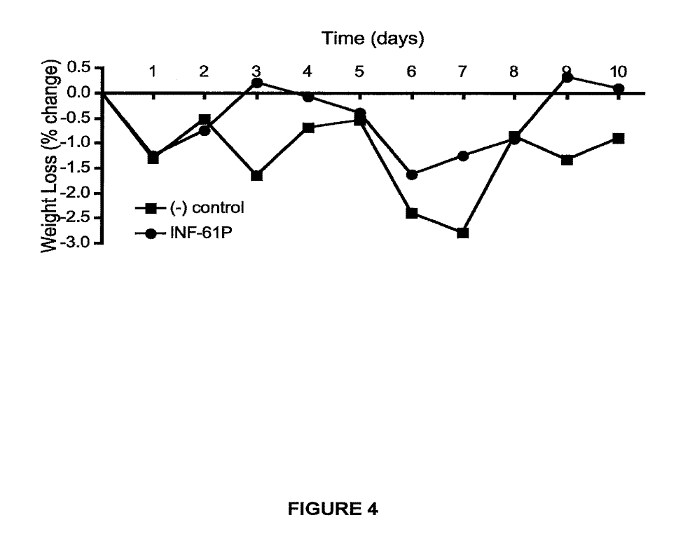

As shown in FIG. 3, when animals were inoculated with immunogenic peptide composition INF-61P (see Example 5) and challenged with H3N2 (A/Wisconsin/2005) virus, they exhibited reduced fever counts, showing a protective effect of composition INF-61P. Furthermore, INF-61P-vaccinated animals (see FIG. 4) exhibited less drastic weight loss in the days after challenge with virus as compared to control mice.

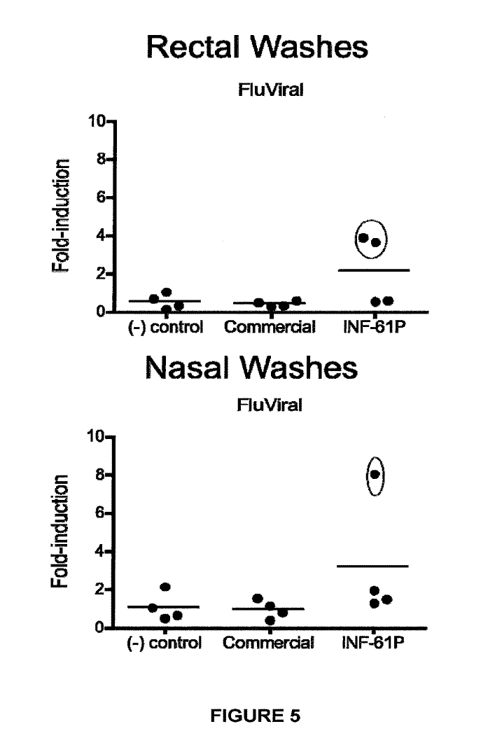

As depicted in FIG. 5, composition INF-61P induced mucosal IgA responses as seen in rectal wash (top panel) and nasal wash (bottom panel) samples. The majority (¾) of animals vaccinated with INF-61P exhibited at least a two-fold increase in IgA titer relative to pre-vaccination responses in either rectal or nasal wash samples. As expected, the commercial vaccine which was given by standard IM injection induced systemic (serum) IgG responses (data not shown). However, as shown in FIG. 5 it induced no IgA responses.

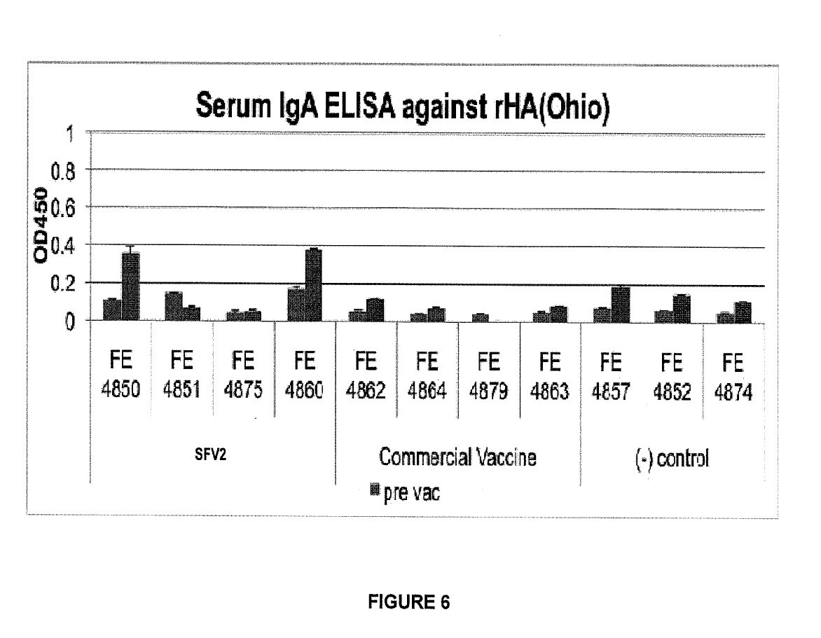

Similar results (data not shown) were observed when this experiment was reproduced with an immunogenic peptide composition that included the SFV2 peptides (see Example 7). Of note, serum IgA directed against recombinant HA protein from influenza type B was detected in animals vaccinated with composition SFV2 (see FIG. 6).

### Example 10

### Immune Response Study of a Subcutaneously Administered INF-09L-A Composition

This Example describes the immune response induced by subcutaneously administering a modified version (INF-09L-A) of the peptide composition of Table 13 (INF-NP1-V1) in mice. The INF-09L-A peptide composition differed from the INF-NP1-V1 composition shown in Table 13 by the inclusion of a KSS linker on the N-terminus of each peptide. The N-terminal lysine moiety was used to double lipidate each peptide.

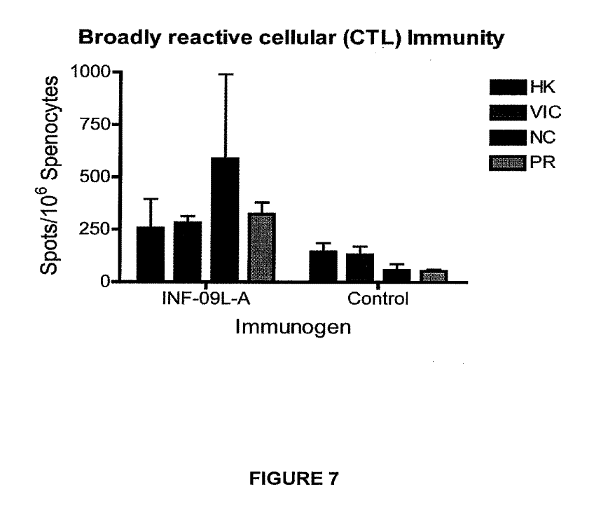

Aged (>12 months) HLA (A*0201) transgenic mice were vaccinated three times with the INF-09L-A peptide composition. All sixteen variants were included in the administered composition. The composition also included an alum adjuvant. A control group only received the adjuvant. Splenocytes were infected with divergent strains of influenza from multiple subtypes: H3N2 (A/HK/1/18, A/VICtoria/3/75) and H1N1 (A/NC/20/99, A/PR/8/34). T cell responses were measured by quantitating the frequency of IFNγ-secreting T cells in an ELISPOT assay (see Example 3 for methodology). Spots represent the frequency of IFNγ-secreting T cells specific for each of the strains of virus, and error bars represent the standard deviation among mice in each group (n=3). The results are presented from left to right as follows: H3N2 (A/HK/1/18), H3N2 (A/VICtoria/3/75), H1N1 (A/NC/20/99) and H1N1 (A/PR/8/34). As shown in FIG. 7, the peptide composition induced a broad cellular (CTL) immune reaction.

### Example 11

### Effect of Adjuvant on Immune Response Induced by an Orally Administered Peptide Composition

This Example compares the immune response induced by orally administering a peptide composition to mice with and without an adjuvant.

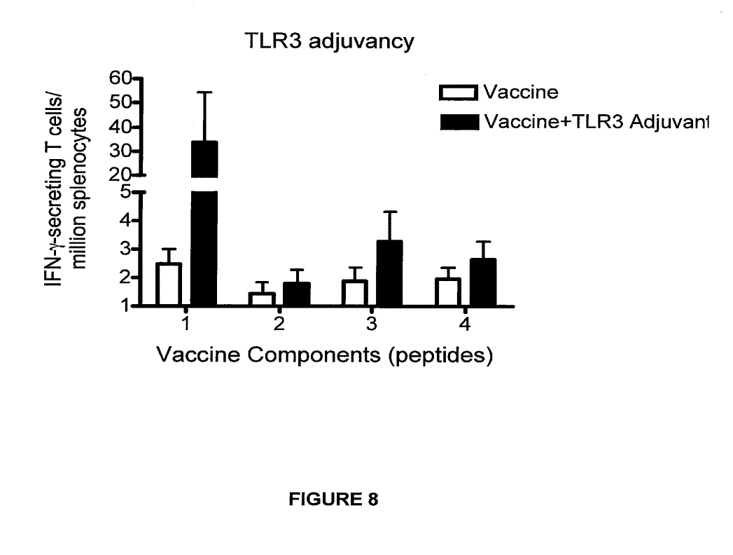

Mice (n=4) received a peptide composition entrapped in bilosomes, either with or without a TLR-3 based adjuvant (poly I:C). Mice were immunized orally 4 times (Days 0, 3, 14, and 17). Splenocytes were harvested after vaccinations and separately stimulated in vitro with the four individual peptides that were present in the peptide composition. Responses were measured using an IFNγ ELISPOT (see Example 3 for methodology). The results for each peptide challenge are compared in FIG. 8. As shown in FIG. 8, animals that received the peptide composition in combination with the adjuvant showed a greater response than did animals that received the peptide composition alone.

### Example 12

### Vesicle SFV2 Composition

This Example describes the preparation of the composition that was used in Example 13 for intramuscular (IM) injections of peptide compositions. The vesicles were composed of the following lipids: 1-monopalmitoyl glycerol (a non-ionic surfactant), cholesterol (a steroid) and dicetylphosphate (an ionic amphiphile). Specifically, a 5:4:1 molar ratio of lipids (270 mg of 1-monopalmitoyl glycerol (MPG), 255 mg of cholesterol (CHO), and 90 mg of dicetylphosphate (DCP)) was placed in a flat bottom 25 ml glass beaker, ensuring none of the powder was adhering to the side of the glass beaker. The beaker was clamped and covered with aluminum foil and the lipids were melted in a heated oil bath at 135° C. for 10 minutes, with occasional swirling in the beaker. In some compositions a synthetic TLR-4 agonist, PHAD (phosphorylated hexaacyl disaccharide from Avanti Polar Lipids, Inc., shown in FIG. 13) was co-melted with the other lipids, in other compositions PHAD was added with the peptide composition SFV2 (described in Example 7). The resulting mixture of 10 ml of peptide composition SFV2 (400 μg/ml) and PHAD (100 μg/ml) was pre-incubated in sodium bicarbonate solution (pH 8.5) for 5 minutes at 30° C. in a heated water bath. The peptide solution was homogenized at 8,000 rpm and then transferred to the melted lipid solution, at which point homogenization continued for 10 minutes at 30° C. (it will be appreciated that alternatively, the heated lipid solution could have been transferred to the antigen solution and subsequently homogenized). Finally, 10 ml of 400 mM sucrose solution in buffer was added to the vesicle/peptide solution and vortexed for 30 seconds. The vesicles (with or without peptide) were aliquoted into vials (0.5 ml/vial) and lyophilized. Lyophilized vesicles (with or without peptide) were rehydrated prior to administration in 0.5 ml of saline and absorbed to alum (aluminum hydroxide).

### Example 13

### Parenteral Immunization of Ferrets with Vesicle SFV2 Composition

This Example describes in vivo testing of the vesicle SFV2 composition of Example 12 in ferrets. For the vaccination and challenge experiments, male ferrets were selected that had been pre-screened as serum negative by the hemagglutination inhibition assay. Compositions for intramuscular injection were formulated as described in Example 12 so that the peptides were contained in a volume of 0.5 ml. All ferrets were bled 3 days before immunization and serum, plasma and PBMCs were collected. Ferrets were immunized by intramuscular injection (IM) with vesicles with or without SFV2 peptide composition on Day 0 and Day 28. For commercial vaccine controls, VAXIGRIP commercial vaccine was injected IM into the quadriceps muscle on Day 0 and Day 28. This commercial vaccine is an inactivated influenza vaccine trivalent for types A and B strains for the 2008-2009 season (specifically strains A/Brisbane/59/2007 H1N1; A/Brisbane/10/2007 H3N2 and B/Florida/4/2006). The experimental groups for this study are summarized in Table 18.

TABLE 18

| | | | | Route of | |
|---|---|---|---|---|---|
| | | Antigen | PHAD | Admin- | No. of |
| Group | Composition | (μg) | (μg) | istration | Animals |
| 1 | Vesicles with SFV2 | 200 | 50 | IM | 4 |
| | peptides, PHAD and | | | | |
| | alum | | | | |
| 3 | VAXIGRIP | 45 | — | IM | 4 |
| 4 | Empty vesicles with | 0 | 50 | IM | 4 |
| | PHAD and alum | | | | |

On Day 28, ferrets were bled and serum samples were collected. All ferrets were then challenged intranasally with influenza virus at 2×10⁵ pfu/ml with PBS (0.5 ml per nostril). For 10 days following virus challenge, nasal washes were collected and all animals were weighed and monitored daily for clinical signs of infection (body temperature measured electronically by implants within each animal). At sacrifice, animals were bled for serum, plasma, PBMCs and (in some instances) complete blood count (CBC), and spleens were collected and processed for mononuclear cells.

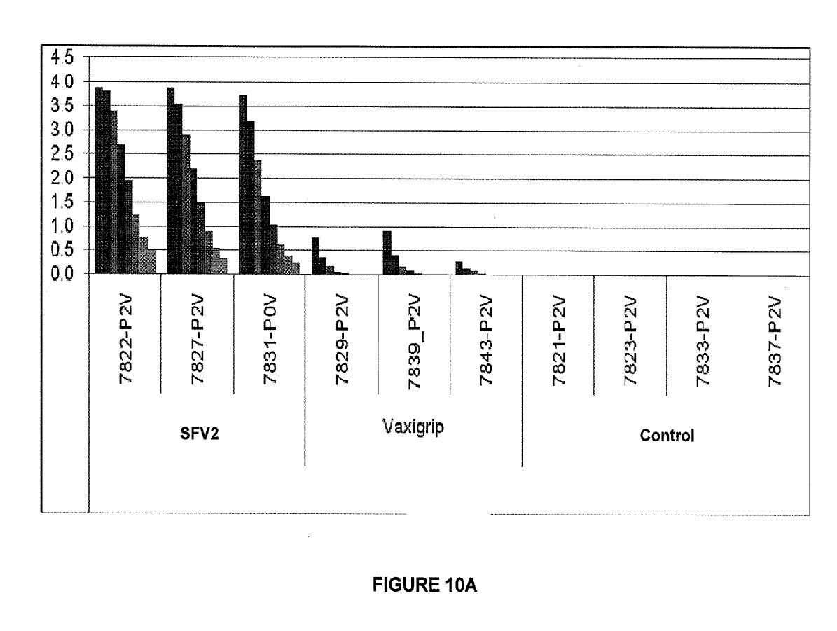

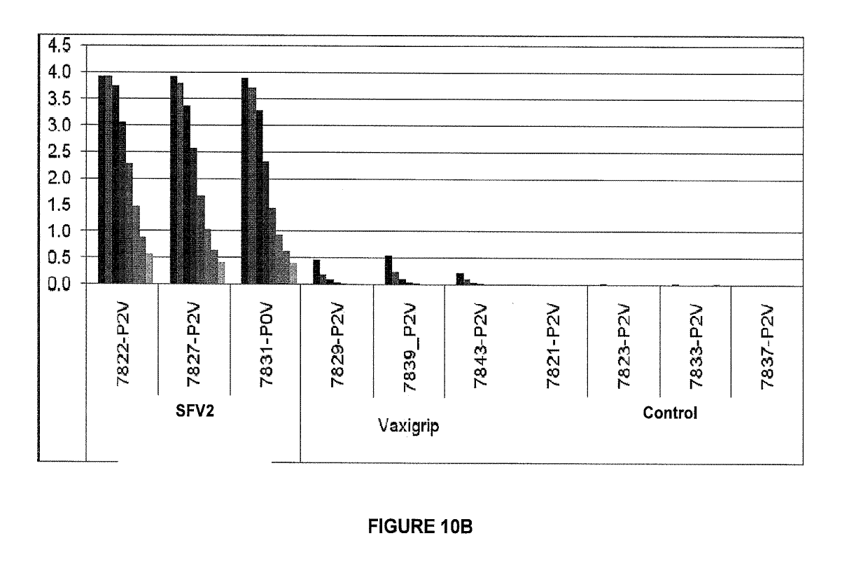

As shown in FIGS. 10A-B, composition SFV2 given IM induces a humoral response. Post immunization serum demonstrated a significant immune response in ELISA with both rHA Solomon (A) and Wisconsin (B) for ferrets immunized with peptide composition SFV2 administered intramuscularly. VAXIGRIP treated animals only showed a small immune response and animals treated with empty vesicles (i.e., without SFV2 peptides) showed no reactivity in ELISA. No IgG response was detected against rHA Malaysia in any of the animal groups (data not shown).

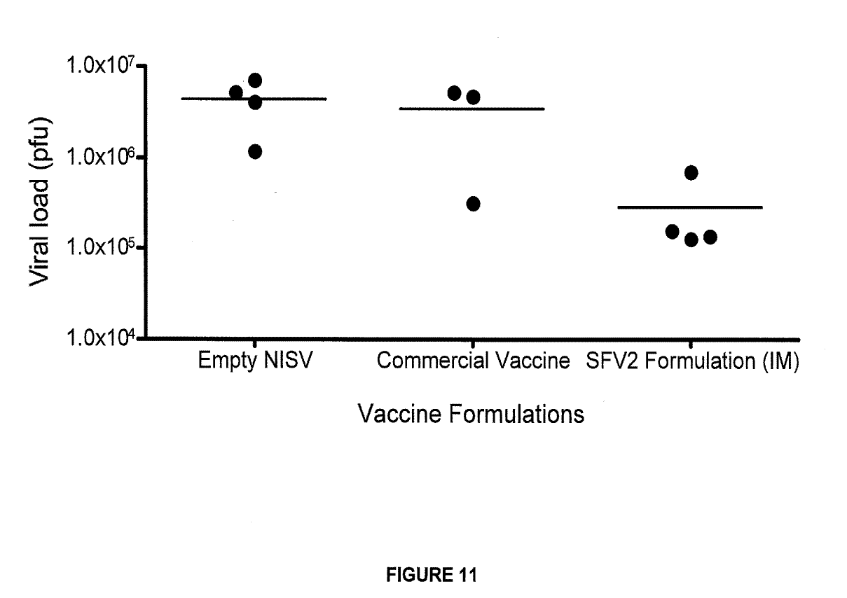

As shown in FIG. 11, composition SFV2 given IM protects against viral challenge. Animals were inoculated with immunogenic peptide composition SFV2 of Example 12 and challenged with 2×10⁵ pfu of H1N1 (A/Solomon Island/03/06) and viral load from nasal wash samples was measured by plaque assay at the peak of viremia (Day 2). Each symbol represents the viral load measured in an individual animal. The animals vaccinated with SFV2 exhibited a significantly reduced (p<0.05) viral load in nasal wash samples compared to control animals treated with empty vesicles. The commercial seasonal influenza vaccine which was also given by standard IM injection did not reduce viral load in nasal wash samples.

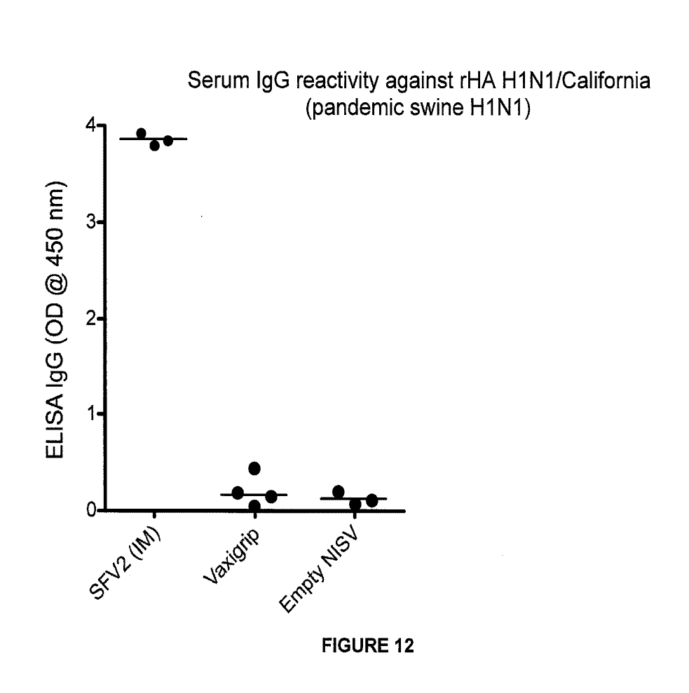

As shown in FIG. 12, composition SFV2 given IM induces humoral immunity against a potential pandemic strain of influenza. Sera was collected from animals two weeks after the second vaccination (prior to challenge) and samples were tested for reactivity with recombinant hemagglutinin (rHA) protein from the putative pandemic swine (H1N1/California/2009) isolate by ELISA. The animals vaccinated with SFV2 exhibited a higher serum IgG reactivity against rHA H1N1/California (pandemic swine H1N1) as compared to control animals treated with empty vesicles. The data in FIG. 12 shows that sera from animals vaccinated with the commercial seasonal influenza vaccine failed to react against this novel strain of influenza.

### Example 14

### Administration Scheme

This Example describes exemplary administration schemes using the vesicle SFV2 composition of Example 12 in humans. A partially-blinded, randomized, placebo-controlled study featuring the following treatment groups is performed:
Treatment Group 1: two intramuscular injections corresponding to the antigen dose 1, vesicle SFV2 composition (1 mg peptide), given on Day 0 and Day 28 (n=50).
Treatment Group 2: two intramuscular injections corresponding to dose 2, SFV2 (0.2 mg peptide) in combination with NAM1, given on Day 0 and Day 28 (n=50).
Treatment Group 3: two intramuscular injections of phosphate buffered saline, placebo group, given on Day 0 and Day 28 (n=25).

Subjects are randomized in a 2:2:1 manner.

Short-term tolerability is assessed on day 7 post-administration. Spontaneously reported adverse events are collected for 28 days post-administration. Serious Adverse Events and Medically Significant Events are collected for a total follow up period of 168 days (±14 days). Biological safety is assessed before and after each injection and at the end of the 168 day follow up period.

Immunogenicity is assessed in serum samples collected on Day 0, Day 28 (±3 days), Day 56 (±7 days) and at the end of the 168 Day (+14 days) follow-up period. The immunological read-outs are serum HI antibodies (Hemagglutination Inhibition assay), rHA-specific serum IgG (ELISA), as well neutralizing antibodies determined by microneutralization assay. Regarding cellular (CTL) immunity evaluation, T-cell response is evaluated by ELISPOT and intracellular cytokine staining.

### Sequence Listing

In accordance with 37 CFR 1.52(e)(5), a Sequence Listing filed herewith in the form of a text file (entitled “Sequence Listing.txt,” created on Jun. 18, 2009, and 14 kilobytes) is incorporated herein by reference in its entirety.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims. The contents of any reference that is referred to herein are hereby incorporated by reference in their entirety.

What is claimed is:

1. An immunogenic composition comprising:
a first peptide comprising a region having at least 80% homology with 20-100 contiguous amino acids of SEQ ID NO. 16, wherein the first peptide includes fewer than 100 contiguous amino acids from a type A influenza hemagglutinin protein;
a second peptide comprising an amino acid sequence of SEQ ID NO. 17; and
a third peptide comprising a region having at least 80% homology with at least 20 contiguous amino acids from positions 2-49 or 68-121 of SEQ ID NO. 18, wherein the third peptide includes fewer than 100 contiguous amino acids from a type B influenza hemagglutinin protein.

2. The composition of claim 1, wherein the first peptide comprises a region having at least 90% homology with 20-100 contiguous amino acids of SEQ ID NO. 16.

3. The composition of claim 1, wherein the first peptide comprises a region having at least 95% homology with 20-100 contiguous amino acids of SEQ ID NO. 16.

4. The composition of claim 1, wherein the first peptide comprises a region having at least 80% homology with 20-50 contiguous amino acids of SEQ ID NO. 16.

5. The composition of claim 1, wherein the first peptide comprises a region having at least 80% homology with 50-100 contiguous amino acids of SEQ ID NO. 16.

6. The composition of claim 1, wherein the first peptide comprises at least 20 contiguous amino acids of SEQ ID NO. 2.

7. The composition of claim 1, wherein the first peptide comprises at least 20 contiguous amino acids of SEQ ID NO. 3.

8. The composition of claim 1, wherein the first peptide comprises at least 20 contiguous amino acids of SEQ TD NO. 4.

9. The composition of claim 1, wherein the first peptide comprises at least 20 contiguous amino acids of SEQ ID NO. 5.

10. The composition of claim 1, wherein the composition comprises 2ⁿ different first peptides each comprising a different amino acid sequence of SEQ ID NO. 2, where n=1-3.

11. The composition of claim 1, wherein the composition comprises 2ⁿ different first peptides each comprising a different amino acid sequence of SEQ ID NO. 3, where n=1-3.

12. The composition of claim 1, wherein the composition comprises 2ⁿ different first peptides each comprising a different amino acid sequence of SEQ ID NO. 4, where n=1-2.

13. The composition of claim 1, wherein the composition comprises 2ⁿ different first peptides each comprising a different amino acid sequence of SEQ ID NO. 5, where n=1-3.

14. The composition of claim 1, wherein the first peptide comprises at least 40 contiguous amino acids of SEQ ID NO. 6.

15. The composition of claim 1, wherein the first peptide comprises at least 60 contiguous amino acids of SEQ ID NO. 6.

16. The composition of claim 1, wherein the first peptide comprises at least 80 contiguous amino acids of SEQ ID NO. 6.

17. The composition of claim 1, wherein the first peptide comprises amino acids 1-81 of SEQ ID NO. 6.

18. The composition of claim 1, wherein the first peptide comprises amino acids 82-88 of SEQ ID NO. 6.

19. The composition of claim 1, wherein the first peptide comprises amino acids 1-88 of SEQ ID NO. 6.

20. The composition of claim 1, wherein the first peptide comprises fewer than 100 amino acids.

21. The composition of claim 1, wherein the second peptide comprises an amino acid sequence of SEQ ID NO. 9.

22. The composition of claim 1, wherein the second peptide comprises an amino acid sequence of SEQ ID NO. 10.

23. The composition of claim 1, wherein the second peptide comprises an amino acid sequence of SEQ ID NO. 11.

24. The composition of claim 1, wherein the composition comprises 2ⁿ different second peptides each comprising a different amino acid sequence of SEQ ID NO. 9, where n=1-4.

25. The composition of claim 1, wherein the composition comprises 2ⁿ different second peptides each comprising a different amino acid sequence of SEQ ID NO. 10, where n=1-4.

26. The composition of claim 1, wherein the composition comprises 2ⁿ different second peptides each comprising a different amino acid sequence of SEQ ID NO. 11, where n=1-4.

27. The composition of any one of claims 24-26, where n=4.

28. The composition of claim 1, wherein the second peptide comprises fewer than 30 amino acids.

29. The composition of claim 1, wherein the third peptide comprises a region having at least 90% homology with at least 20 contiguous amino acids from positions 2-49 or 68-121 of SEQ ID NO. 18.

30. The composition of claim 1, wherein the third peptide comprises a region having at least 80% homology with at least 40 contiguous amino acids from positions 2-49 or 68-121 of SEQ ID NO. 18.

31. The composition of claim 1, wherein the third peptide comprises a first region having at least 80% homology with at least 20 contiguous amino acids from positions 2-49 and a second region having at least 80% homology with at least 20 contiguous amino acids from positions 68-121 of SEQ ID NO. 18.

32. The composition of claim 1, wherein the third peptide comprises a region having at least 80% homology with SEQ ID NO. 8.

33. The composition of claim 1, wherein the third peptide comprises a region having at least 90% homology with SEQ ID NO. 8.

34. The composition of claim 1, wherein the third peptide comprises a region having at least 95% homology with SEQ ID NO. 8.

35. The composition of claim 1, wherein the composition comprises two different third peptides, one of which comprises a region having at least 80% homology with at least 20 contiguous amino acids from positions 2-49 of SEQ ID NO. 18 while the other comprises a region having at least 80% homology with at least 20 contiguous amino acids from positions 68-121 of SEQ ID NO. 18.

36. The composition of claim 1, wherein the third peptide comprises fewer than 100 amino acids.

37. The composition of claim 1, wherein the composition further comprises:
a fourth peptide comprising an amino acid sequence of SEQ ID NO. 14, wherein the fourth peptide includes fewer than 20 contiguous amino acids from a type A influenza nucleoprotein.

38. The composition of claim 37, wherein the composition comprises 2ⁿ different fourth peptides each comprising a different amino acid sequence of SEQ ID NO. 14, where n=1-4.

39. The composition of claim 38, where n=4.

40. The composition of claim 37, wherein the fourth peptide comprises fewer than 30 amino acids.

41. The composition of claim 1, wherein the composition further comprises:
a fourth peptide comprising an amino acid sequence of SEQ ID NO. 15, wherein the fourth peptide includes fewer than 20 contiguous amino acids from a type A influenza nucleoprotein.

42. The composition of claim 41, wherein the composition comprises 2ⁿ different fourth peptides each comprising a different amino acid sequence of SEQ ID NO. 15, where n=1-4.

43. The composition of claim 42, where n=4.

44. The composition of claim 41, wherein the fourth peptide comprises fewer than 30 amino acids.

45. The composition of claim 1, wherein the first peptide comprises a region having at least 80% homology with SEQ ID NO. 6, the third peptide comprises a region having at least 80% homology with SEQ ID NO. 8 and the composition comprises 2ⁿ different second peptides each comprising a different amino acid sequence of SEQ ID NO. 11, where n=1-4.

46. The composition of claim 1, wherein the first peptide comprises a region having at least 90% homology with SEQ ID NO. 6, the third peptide comprises a region having at least 90% homology with SEQ ID NO. 8 and the composition comprises 2ⁿ different second peptides each comprising a different amino acid sequence of SEQ ID NO. 11, where n=1-4.

47. The composition of claim 1, wherein the first peptide comprises a region having at least 95% homology with SEQ ID NO. 6, the third peptide comprises a region having at least 95% homology with SEQ ID NO. 8 and the composition comprises 2ⁿ different second peptides each comprising a different amino acid sequence of SEQ ID NO. 11, where n=1-4.

48. The composition of claim 1, wherein the first peptide comprises SEQ ID NO. 6, the third peptide comprises SEQ ID NO. 8 and the composition comprises 2ⁿ different second peptides each comprising a different amino acid sequence of SEQ ID NO. 11, where n=1-4.

49. The composition of any one of claims 45-48, where n=4.

50. The composition of claim 1, wherein the first peptide consists essentially of SEQ ID NO. 6, the third peptide consists essentially of SEQ ID NO. 8 and the composition comprises 2ⁿ different second peptides each consisting essentially of a different amino acid sequence of SEQ ID NO. 11, where n=1-4.

51. The composition of claim 50, wherein the composition further comprises 2^{m} different fourth peptides each comprising a different amino acid sequence of SEQ ID NO. 14, where m=1-4.

52. The composition of claim 51, wherein n and/or m=2.

53. The composition of claim 51, wherein n and/or m=3.

54. The composition of claim 51, wherein n and/or m=4.

55. The composition of any one of the preceding claims, wherein each peptide is immunogenic.

56. The composition of any one of the preceding claims, wherein at least one peptide further comprises a region with 20 or more contiguous amino acids from a non-influenza protein.

57. The composition of any one of the preceding claims, wherein each peptide further comprises a region with 20 or more contiguous amino acids from a non-influenza protein.

58. The composition of any one of the preceding claims, wherein at least two peptides are present within a single protein.

59. The composition of any one of the preceding claims, wherein the first and third peptides comprise fewer than 100 amino acids and the second peptide comprises fewer than 30 amino acids.

60. The composition of any one of the preceding claims, wherein the composition further comprises an adjuvant.

61. The composition of any one of the preceding claims, wherein the adjuvant is alum.

62. The composition of any one of the preceding claims, wherein the adjuvant is an immunologically active saponin fraction having adjuvant activity derived from the bark of the South American tree Quillaja Saponaria Molina.

63. The composition of any one of the preceding claims, wherein the adjuvant is a TLR-3 agonist.

64. The composition of claim 63, wherein the adjuvant is
polyriboinosinic:polyribocytidylic acid.

65. The composition of any one of the preceding claims, wherein the adjuvant is a TLR-4 agonist.

66. The composition of claim 65, wherein the adjuvant contains monophosphoryl lipid A or 3-deacyl monophosphoryl lipid A.

67. The composition of any one of the preceding claims, wherein the adjuvant is a TLR-7/8 agonist.

68. The composition of claim 67, wherein the adjuvant is 1-isobutyl-1H-imidazo[4,5-c]quinolin-4-amine.

69. The composition of any one of the preceding claims, wherein at least one peptide is associated with a vesicle.

70. The composition of claim 69, wherein the vesicle comprises a non-ionic surfactant.

71. The composition of claim 70, wherein the vesicle comprises a glycerol ester.

72. The composition of claim 70, wherein the vesicle comprises a glycol or glycerol ether.

73. The composition of claim 70, wherein the vesicle comprises a transport enhancer which facilitates the transport of lipid-like molecules across mucosal membranes.

74. The composition of claim 73, wherein the vesicle comprises a cholesterol derivatives in which the C₂₃ carbon atom of the side chain carries a carboxylic acid.

75. The composition of claim 73, wherein the vesicle comprises cholic acid, chenodeoxycholic acid or a salt thereof.

76. The composition of claim 73, wherein the vesicle comprises glycocholic acid, taurocholic acid, deoxycholicacid, ursodeoxycholic acid, or a salt thereof.

77. The composition of claim 73, wherein the vesicle comprises an acyloxylated amino acid or a salt thereof.

78. The composition of claim 73, wherein the vesicle comprises an acylcarnitine containing a C₆₋₂₀alkanoyl or alkenoyl moiety or a salt thereof.

79. The composition of claim 73, wherein the vesicle comprises an ionic surfactant.

80. The composition of claim 73, wherein the vesicle comprises alkanoicoralkenoic acid.

81. The composition of claim 73, wherein the vesicle comprises a phosphate.

82. The composition of claim 73, wherein the vesicle comprises dicetylphospate, phosphatidic acid or phosphatidyl serine.

83. The composition of claim 73, wherein the vesicle comprises a sulphate monoester.

84. The composition of claim 73, wherein the vesicle comprises cetylsulphate.

85. The composition of claim 73, wherein the vesicle comprises a steroid.

86. The composition of claim 73, wherein the vesicle comprises cholesterol.

87. The composition of claim 73, wherein the vesicle has a diameter in the range of about 10 nm to about 10 μm.

88. The composition of claim 73, wherein the vesicle has a diameter in the range of about 800 nm to about 1.5 μm.

89. The composition of claim 73, wherein at least one peptide is encapsulated within an aqueous core of the vesicle.

90. An immunogenic composition comprising a peptide comprising a region having at least 80% homology with 20-100 contiguous amino acids of SEQ ID NO. 16, wherein the peptide includes fewer than 100 contiguous amino acids from a type A influenza hemagglutinin protein.

91. The composition of claim 90, wherein the peptide comprises at least 20 contiguous amino acids of SEQ ID NO. 2.

92. The composition of claim 90, wherein the peptide comprises at least 20 contiguous amino acids of SEQ ID NO. 3.

93. The composition of claim 90, wherein the peptide comprises at least 20 contiguous amino acids of SEQ ID NO. 4.

94. The composition of claim 90, wherein the peptide comprises at least 20 contiguous amino acids of SEQ ID NO. 5.

95. The composition of claim 90, wherein the composition comprises 2ⁿ different peptides each comprising a different amino acid sequence of SEQ ID NO. 2, where n=1-3.

96. The composition of claim 90, wherein the composition comprises 2ⁿ different peptides each comprising a different amino acid sequence of SEQ ID NO. 3, where n=1-3.

97. The composition of claim 90, wherein the composition comprises 2ⁿ different peptides each comprising a different amino acid sequence of SEQ ID NO. 4, where n=1-2.

98. The composition of claim 90, wherein the composition comprises 2ⁿ different peptides each comprising a different amino acid sequence of SEQ ID NO. 5, where n=1-3.

99. The composition of claim 90, wherein the peptide comprises at least 40 contiguous amino acids of SEQ ID NO. 6.

100. The composition of claim 90, wherein the peptide comprises at least 60 contiguous amino acids of SEQ ID NO. 6.

101. The composition of claim 90, wherein the peptide comprises at least 80 contiguous amino acids of SEQ ID NO. 6.

102. The composition of claim 90, wherein the peptide comprises a region having at least 90% homology with SEQ ID NO. 6.

103. The composition of claim 90, wherein the peptide comprises a region having at least 95% homology with SEQ ID NO. 6.

104. The composition of claim 90, wherein the peptide comprises amino acids 1-81 of SEQ ID NO. 6.

105. The composition of claim 90, wherein the peptide comprises amino acids 82-88 of SEQ ID NO. 6.

106. The composition of claim 90, wherein the peptide comprises SEQ ID NO. 6.

107. The composition of claim 90, wherein the peptide consists essentially of SEQ ID NO. 6.

108. The composition of claim 90, wherein the peptide consists of SEQ ID NO. 6.

109. The composition of claim 90, wherein the peptide comprises fewer than 30 amino acids.

110. An immunogenic composition comprising a peptide comprising an amino acid sequence of SEQ ID NO. 17.

111. The composition of claim 110, wherein the peptide comprises an amino acid sequence of SEQ ID NO. 9.

112. The composition of claim 110, wherein the peptide comprises an amino acid sequence of SEQ ID NO. 10.

113. The composition of claim 110, wherein the peptide comprises an amino acid sequence of SEQ ID NO. 11.

114. The composition of claim 110, wherein the composition comprises 2ⁿ different peptides each comprising a different amino acid sequence of SEQ ID NO. 9, where n=1-4.

115. The composition of claim 110, wherein the composition comprises 2ⁿ different peptides each comprising a different amino acid sequence of SEQ ID NO. 10, where n=1-4.

116. The composition of claim 110, wherein the composition comprises 2ⁿ different peptides each comprising a different amino acid sequence of SEQ ID NO. 11, where n=1-4.

117. The composition of claim 116, wherein the composition comprises 2ⁿ different peptides each consisting essentially of a different amino acid sequence of SEQ ID NO. 11.

118. The composition of any one of claims 114-117, where n=4.

119. The composition of claim 110, wherein the peptide comprises fewer than 30 amino acids.

120. An immunogenic composition comprising a peptide comprising a region having at least 80% homology with at least 20 contiguous amino acids from positions 2-49 or 68-121 of SEQ ID NO. 18, wherein the peptide includes fewer than 100 contiguous amino acids from a type B influenza hemagglutinin protein.

121. The composition of claim 120, wherein the peptide comprises a region having at least 90% homology with at least 20 contiguous amino acids from positions 2-49 or 68-121 of SEQ ID NO. 18.

122. The composition of claim 120, wherein the peptide comprises a region having at least 80% homology with at least 40 contiguous amino acids from positions 2-49 or 68-121 of SEQ ID NO. 18.

123. The composition of claim 120, wherein the peptide comprises a first region having at least 80% homology with at least 20 contiguous amino acids from positions 2-49 and a second region having at least 80% homology with at least 20 contiguous amino acids from positions 68-121 of SEQ ID NO. 18.

124. The composition of claim 120, wherein the peptide comprises a region having at least 80% homology with SEQ ID NO. 8.

125. The composition of claim 120, wherein the peptide comprises a region having at least 90% homology with SEQ ID NO. 8.

126. The composition of claim 120, wherein the peptide comprises a region having at least 95% homology with SEQ ID NO. 8.

127. The composition of claim 120, wherein the peptide comprises SEQ ID NO. 8.

128. The composition of claim 120, wherein the peptide consists essentially of SEQ ID NO. 8.

129. The composition of claim 120, wherein the peptide consists of SEQ ID NO. 8.

130. The composition of claim 120, wherein the peptide comprises a region having at least 80% homology with at least 20 contiguous amino acids from positions 2-49 of SEQ ID NO. 18 and the composition further comprises a different peptide comprising a region having at least 80% homology with at least 20 contiguous amino acids from positions 68-121 of SEQ ID NO. 18.

131. The composition of claim 120, wherein the peptide comprises fewer than 100 amino acids.

132. An immunogenic composition comprising a peptide comprising an amino acid sequence of SEQ ID NO. 14, wherein the peptide includes fewer than 20 contiguous amino acids from a type A influenza nucleoprotein.

133. The composition of claim 132, wherein the composition comprises 2ⁿ different peptides each comprising a different amino acid sequence of SEQ ID NO. 14, where n=1-4.

134. The composition of claim 133, where n=4.

135. The composition of claim 132, wherein the peptide comprises fewer than 30 amino acids.

136. An immunogenic composition comprising a peptide comprising an amino acid sequence of SEQ ID NO. 15, wherein the peptide includes fewer than 20 contiguous amino acids from a type A influenza nucleoprotein.

137. The composition of claim 136, wherein the composition comprises 2ⁿ different peptides each comprising a different amino acid sequence of SEQ ID NO. 15, where n=1-4.

138. The composition of claim 137, where n=4.

139. The composition of claim 136, wherein the peptide comprises fewer than 30 amino acids.

140. An immunogenic composition comprising a peptide comprising an amino acid sequence of SEQ ID NO. 1.

141. The composition of claim 140, wherein the composition comprises 2ⁿ different peptides each comprising a different amino acid sequence of SEQ ID NO. 1, where n=1-4.

142. The composition of claim 141, where n=4.

143. The composition of claim 140, wherein the peptide comprises fewer than 30 amino acids.

144. An immunogenic composition comprising a peptide comprising a region having at least 80% homology with at least 20 contiguous amino acids of SEQ ID NO. 7, wherein the peptide includes fewer than 100 contiguous amino acids from a type A influenza hemagglutinin protein.

145. The composition of claim 144, wherein the region has at least 90% homology with a region of SEQ ID NO. 7.

146. The composition of claim 144, wherein the region has at least 95% homology with a region of SEQ ID NO. 7.

147. The composition of claim 144, wherein the peptide comprises at least 40 contiguous amino acids of SEQ ID NO. 7.

148. The composition of claim 144, wherein the peptide comprises at least 60 contiguous amino acids of SEQ ID NO. 7.

149. The composition of claim 144, wherein the peptide comprises at least 80 contiguous amino acids of SEQ ID NO. 7.

150. The composition of claim 144, wherein the peptide comprises SEQ ID NO. 7.

151. The composition of claim 144, wherein the peptide consists essentially of SEQ ID NO. 7.

152. The composition of claim 144, wherein the peptide consists of SEQ ID NO. 7.

153. The composition of claim 144, wherein the peptide comprises fewer than 100 amino acids.

154. A method of treating an individual suffering from, or at risk for, influenza, the method comprising administering to the individual a therapeutically effective amount of the composition of any one of the previous claims.

155. The method of claim 154, wherein the composition is administered orally.

156. The method of claim 154, wherein the composition is administered parenterally.

157. The method of claim 154, wherein the composition is administered by intramuscular injection.

158. The method of claim 154, wherein the composition is administered intranasally or by inhalation.

159. The method of claim 154, wherein the composition is administered rectally.

160. The method of claim 154, wherein the individual is suffering from, or at risk for, seasonal influenza.

161. The method of claim 154, wherein the individual is suffering from, or at risk for, influenza caused by an influenza A (H1N1) virus of swine origin.

162. An immunogenic composition comprising:
a TLR-3 agonist adjuvant;
a vesicle which comprises a non-ionic surfactant and a transport enhancer which facilitates the transport of lipid-like molecules across mucosal membranes;
a first peptide comprising a region having at least 80% homology with 20-100 contiguous amino acids of SEQ ID NO. 16, wherein the first peptide includes fewer than 100 contiguous amino acids from a type A influenza hemagglutinin protein;
a second peptide comprising an amino acid sequence of SEQ ID NO. 17;
a third peptide comprising a region having at least 80% homology with at least 20 contiguous amino acids from positions 2-49 or 68-121 of SEQ ID NO. 18, wherein the third peptide includes fewer than 100 contiguous amino acids from a type B influenza hemagglutinin protein; and
a fourth peptide comprising an amino acid sequence of SEQ ID NO. 14, wherein the fourth peptide includes fewer than 20 contiguous amino acids from a type A influenza nucleoprotein.

163. The composition of claim 162, wherein the TLR-3 agonist adjuvant comprises poly(I:C).

164. The composition of any one of claims 162-163, wherein the transport enhancer is a bile acid, a derivative thereof or a salt of any of these.

165. The composition of claim 164, wherein the transport enhancer is sodium deoxycholate.

166. The composition of any one of claims 162-165, wherein the non-ionic surfactant is a glycerol ester.

167. The composition of claim 166, wherein the non-ionic surfactant is 1-monopalmitoyl glycerol.

168. The composition of any one of claims 162-167, wherein the vesicle further comprises an ionic amphiphile.

169. The composition of claim 168, wherein the ionic amphiphile is dicetylphospate.

170. The composition of any one of claims 162-169, wherein the vesicle further comprises a steroid.

171. The composition of claim 170, wherein the steroid is cholesterol.

172. The composition of claim 162, wherein the vesicle comprises 1-monopalmitoyl glycerol, dicetylphospate, cholesterol and sodium deoxycholate.

173. The composition of any one of claims 162-172, wherein the composition further comprises alum.

174. A method of treating an individual suffering from, or at risk for, influenza, the method comprising orally administering to the individual a therapeutically effective amount of the composition of any one of claims 162-173.

175. An immunogenic composition comprising:
a TLR-4 agonist adjuvant;
a vesicle which comprises a non-ionic surfactant;
a first peptide comprising a region having at least 80% homology with 20-100 contiguous amino acids of SEQ ID NO. 16, wherein the first peptide includes fewer than 100 contiguous amino acids from a type A influenza hemagglutinin protein;
a second peptide comprising an amino acid sequence of SEQ ID NO. 17;
a third peptide comprising a region having at least 80% homology with at least 20 contiguous amino acids from positions 2-49 or 68-121 of SEQ ID NO. 18, wherein the third peptide includes fewer than 100 contiguous amino acids from a type B influenza hemagglutinin protein; and
a fourth peptide comprising an amino acid sequence of SEQ ID NO. 14, wherein the fourth peptide includes fewer than 20 contiguous amino acids from a type A influenza nucleoprotein.

176. The composition of claim 175, wherein the TLR-4 agonist adjuvant comprises monophosphoryl lipid A or 3-deacyl monophosphoryl lipid A.

177. The composition of any one of claims 175-176, wherein the non-ionic surfactant is a glycerol ester.

178. The composition of claim 177, wherein the non-ionic surfactant is 1-monopalmitoyl glycerol.

179. The composition of any one of claims 175-178, wherein the vesicle further comprises an ionic amphiphile.

180. The composition of claim 179, wherein the ionic amphiphile is dicetylphospate.

181. The composition of any one of claims 175-180, wherein the vesicle further comprises a steroid.

182. The composition of claim 181, wherein the steroid is cholesterol.

183. The composition of claim 175, wherein the vesicle comprises 1-monopalmitoyl glycerol, dicetylphospate, cholesterol and sodium deoxycholate.

184. The composition of any one of claims 175-183, wherein the composition further comprises alum.

185. A method of treating an individual suffering from, or at risk for, influenza, the method comprising parenterally administering to the individual a therapeutically effective amount of the composition of any one of claims 175-184.

186. The method of claim 185, wherein the composition is administered by intramuscular injection.

187. The composition of any one of claim 162-173 or 175-184, wherein the first peptide comprises at least 40 contiguous amino acids of SEQ ID NO. 6.

188. The composition of any one of claim 162-173 or 175-184, wherein the first peptide comprises amino acids 1-88 of SEQ ID NO. 6.

189. The composition of any one of claim 162-173 or 175-184, wherein the second peptide comprises an amino acid sequence of SEQ ID NO. 11.

190. The composition of any one of claim 162-173 or 175-184, wherein the composition comprises 2ⁿ different second peptides each comprising a different amino acid sequence of SEQ ID NO. 11, where n=1-4.

191. The composition of claim 190, where n=4.

192. The composition of any one of claim 162-173 or 175-184, wherein the third peptide comprises a region having at least 90% homology with at least 20 contiguous amino acids from positions 2-49 or 68-121 of SEQ ID NO. 18.

193. The composition of any one of claim 162-173 or 175-184, wherein the third peptide comprises a first region having at least 80% homology with at least 20 contiguous amino acids from positions 2-49 and a second region having at least 80% homology with at least 20 contiguous amino acids from positions 68-121 of SEQ ID NO. 18.

194. The composition of any one of claim 162-173 or 175-184, wherein the composition comprises two different third peptides, one of which comprises a region having at least 80% homology with at least 20 contiguous amino acids from positions 2-49 of SEQ ID NO. 18 while the other comprises a region having at least 80% homology with at least 20 contiguous amino acids from positions 68-121 of SEQ ID NO. 18.

195. The composition of any one of claim 162-173 or 175-184, wherein the composition comprises two different third peptides, one of which comprises an amino acid sequence of SEQ ID NO. 12 while the other comprises an amino acid sequence of SEQ ID NO. 13.

196. The composition of any one of claim 162-173 or 175-184, wherein the composition comprises 2ⁿ different fourth peptides each comprising a different amino acid sequence of SEQ ID NO. 14, where n=1-4.

197. The composition of claim 196, where n=4.

198. The composition of any one of claim 162-173 or 175-184, wherein:
the first peptide comprises amino acids 1-88 of SEQ ID NO. 6.
the composition comprises 2ⁿ different second peptides each comprising a different amino acid sequence of SEQ ID NO. 11, where n=4;
the composition comprises two different third peptides, one of which comprises an amino acid sequence of SEQ ID NO. 12 while the other comprises an amino acid sequence of SEQ ID NO. 13; and
the composition comprises 2^{m} different fourth peptides each comprising a different amino acid sequence of SEQ ID NO. 14, where m=4.


SEQUENCE LISTING (18 sequences)

SEQ ID NO: 1 - protein, 28 aa, Artificial Sequence
  Immunogenic influenza peptide
GSRPXVREDGGLPQSXRISIXWTIVKPG

SEQ ID NO: 2 - protein, 26 aa, Artificial Sequence
  Immunogenic influenza peptide
XTGVSASCSHNGXSSFYXNLLWLTGK

SEQ ID NO: 3 - protein, 33 aa, Artificial Sequence
  Immunogenic influenza peptide
HYSRXFTPEIXKRPKVRXQEGRINYYWTLLEPG

SEQ ID NO: 4 - protein, 30 aa, Artificial Sequence
  Immunogenic influenza peptide
PVTIGECPKYVRSXKLRMXTGLRNIPSIQS

SEQ ID NO: 5 - protein, 29 aa, Artificial Sequence
  Immunogenic influenza peptide
CELLISXESWSYIVEXPNPENGTCYPGXF

SEQ ID NO: 6 - protein, 88 aa, Artificial Sequence
  Immunogenic influenza peptide
SWPNHTTTGVSASSSHNGESSFYKNLLWLTGKNGLYPNLSKSYANNKEKEVLVLWGVHHPPNIGDQRALYHKENAYVSVVSIIFEANG

SEQ ID NO: 7 - protein, 87 aa, Artificial Sequence
  Immunogenic influenza peptide
NWTGVTQNGTSSSSKRRSNNSFFSRLNWLTHLKFKYPALNTMPNNEKFDKLYIWGVHHPVTDNDQIFLYAQASGRITVSTLLINSTG

SEQ ID NO: 8 - protein, 98 aa, Artificial Sequence
  Immunogenic influenza peptide
NAEKAPGGPYKIGTSGSSPNVTNGNGFFATMAWAVPKNDNNKTATNSLTIEVPYISTEGEDQITIWGFHSDNETQMAKLYGDSKPQKFTSSAGTITYQ

SEQ ID NO: 9 - protein, 21 aa, Artificial Sequence
  Immunogenic influenza peptide
YACKXGGKSSGSSYPVLXXXY

SEQ ID NO: 10 - protein, 21 aa, Artificial Sequence
  Immunogenic influenza peptide
YACKXGGKSSGSSYPVLXVXX

SEQ ID NO: 11 - protein, 21 aa, Artificial Sequence
  Immunogenic influenza peptide
YASKXGGKSSGSSYPVLXVXX

SEQ ID NO: 12 - protein, 48 aa, Artificial Sequence
  Immunogenic influenza peptide
AEKAPGGPYKIGTSGSSPNVTNGNGFFATMAWAVPKNDNNKTATNSLT

SEQ ID NO: 13 - protein, 54 aa, Artificial Sequence
  Immunogenic influenza peptide
FHSDNETQMAKLYGDSKPQKFTSSANGVTTHYVSQIGGFPNQTEDGGLPQSGRI

SEQ ID NO: 14 - protein, 15 aa, Artificial Sequence
  Immunogenic influenza peptide
SVQRNLPFXXXTXMA

SEQ ID NO: 15 - protein, 16 aa, Artificial Sequence
  Immunogenic influenza peptide
XXXSSTLELRSXYWAI

SEQ ID NO: 16 - protein, 180 aa, Artificial Sequence
  Immunogenic influenza peptide
CELLISRESWSYIVEKPNPENGTCYPGHFSWPNHTTTGVSASCSHNGESSFYKNLLWLTGKNGLYPNLSKSYANNKEKEVLVLWGVHHPPNIGDQRALYHKENAYVSVVSHYSRKFTPEIAKRPKVRDQEGRINYYWTLLEPGIIFEANGPVTIGECPKYVRSAKLRMVTGLRNIPSIQS

SEQ ID NO: 17 - protein, 21 aa, Artificial Sequence
  Immunogenic influenza peptide
YAXKXGGKSSGSSYPVLXXXX

SEQ ID NO: 18 - protein, 127 aa, Artificial Sequence
  Immunogenic influenza peptide
NAEKAPGGPYKIGTSGSSPNVTNGNGFFATMAWAVPKNDNNKTATNSLTIEVPYISTEGEDQITIWGFHSDNETQMAKLYGDSKPQKFTSSANGVTTHYVSQIGGFPNQTEDGGLPQSGRIGTITYQ